(19) United States
(12) Patent Application Publication
(10) Pub. No.: US 2024/0366183 A1
(43) Pub. Date: Nov. 7, 2024

(54) REAL-TIME SUPER-RESOLUTION ULTRASOUND MICROVESSEL IMAGING AND VELOCIMETRY

(71) Applicant: The Board of Trustees of the University of Illinois, Urbana, IL (US)

(72) Inventors: Pengfei Song, Champaign, IL (US); Xi Chen, Champaign, IL (US); Matthew R. Lowerison, Champaign, IL (US)

(21) Appl. No.: 18/690,239

(22) PCT Filed: Sep. 1, 2022
(86) PCT No.: PCT/US22/75864

§ 371 Date: Mar. 7, 2024

Related U.S. Application Data
(60) Provisional application No. 63/242,022, filed on Sep. 8, 2021.

(51) Int. Cl. A61B 8/08

(52) U.S. Cl. CPC A61B 8/0891; A61B 8/481; A61B 8/5207

(57) ABSTRACT

Described here are systems and methods for super-resolution ultrasound microvessel imaging and velocimetry. The systems and methods utilize deep learning and parallel computing to realize real-time super-resolution microvascular imaging and quantitative analysis and display.

### STATEMENT REGARDING FEDERALLY SPONSORED RESEARCH

This invention was made with government support under EB030072 and CA214523 awarded by the National Institutes of Health. The government has certain rights in the invention.

### BACKGROUND

An enduring goal for biomedical imaging is the creation of a noninvasive microvascular imaging modality that offers deep imaging penetration and exquisite spatial resolution. To date, a multitude of imaging technologies have been developed, which span a wide spectrum ranging from electromagnetic radiation to acoustic waves. However, very few of these imaging modalities provide high imaging resolution and deep imaging penetration at the same time.

The super-resolution ultrasound microvessel imaging (“SR-UMI”) technique has shown great potential in solving the resolution-penetration conundrum. Leveraging the widely-used ultrasound contrast microbubbles with super-resolution imaging strategies similar to Photoactivated Localization Microscopy (“PALM”) and Stochastic Optical Reconstruction Microscopy (“STORM”), SR-UMI improves ultrasound imaging resolution by approximately 10-fold while preserving the imaging penetration. Not only can SR-UMI resolve capillary-scale blood vessels at clinically relevant imaging depth (>10 cm), but it also can accurately measure microvascular blood flow speeds as low as 1 mm/s. As such, SR-UMI effectively extends an optical imaging resolution to the penetration depth of acoustics, while providing structural (e.g., microvessel density, tortuosity) and functional (e.g., microvessel blood flow speed, inter-vessel distance) information of tissue microvasculature. In addition, as an ultrasound-based imaging technique, SR-UMI is noninvasive, low-cost, widely accessible, and does not use ionizing radiation. These unique functionalities and features of SR-UMI have gained traction rapidly and have opened new doors for a wide range of clinical applications including diagnosis and characterization of cancer, cardiovascular diseases, diabetes, and neurodegenerative diseases such as Alzheimer's disease.

Despite the promising clinical potential, SR-UMI is not without its drawbacks. Future clinical translation of SR-UMI is hampered by technical barriers, including slow data acquisition and computationally expensive post-processing. On the one hand, SR-UMI data acquisition is very slow. Currently, the temporal resolution of SR-UMI is limited by the long data acquisition time needed to accumulate adequate microbubble signal, which typically takes tens of seconds. Such long data acquisition requires long breath-hold from patients, precludes the possibility of real-time imaging or imaging on animal models without controlled breathing, and is not suited for capturing the fast hemodynamic properties of tissue. SR-UMI post-processing is also very computationally expensive. Due to the complexity of microbubble localization and tracking algorithms and the sheer amount of data associated with ultrafast plane wave imaging, generating a single two-dimensional (“2D”) SR-UMI image can take hours of data processing.

### SUMMARY OF THE DISCLOSURE

The present disclosure addresses the aforementioned drawbacks by providing a method for super-resolution microvessel imaging using an ultrasound system. The method includes acquiring ultrasound signal data (e.g., microbubble signal data) from a subject using the ultrasound system. A neural network that has been trained on training data to estimate at least one of super-resolution microvessel image data or super-resolution ultrasound velocimetry data from ultrasound signals is then accessed with a computer system. The ultrasound signal data are input to the neural network, via the computer system, generating output data as at least one of super-resolution microvessel image data or super-resolution ultrasound velocimetry data. The super-resolution microvessel image data and/or super-resolution ultrasound velocimetry data are then provided to a user via the computer system.

It is another aspect of the present disclosure to provide a method for training a neural network for super-resolution ultrasound microvessel imaging. The method includes providing a chorioallantoic membrane (CAM) assay for imaging. Simultaneous imaging of the CAM assay is performed with an optical imaging system and an acoustic imaging system, generating optical CAM assay image data and acoustic CAM assay image data. A training data set is assembled with a computer system based on the optical CAM assay image data and the acoustic CAM assay image data. A neural network is then trained using the training data set.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 illustrates a system for ultrasound imaging according to an embodiment of the present disclosure.

FIG. 2 illustrates modules/components of an example real-time super-resolution imaging processing unit according to an embodiment of the present disclosure.

FIG. 3 is a flow chart illustrating steps for constructing a neural network according to an embodiment of the present disclosure.

FIG. 4 shows an example of an experimentally determined vascular structure map based on an x-ray CT image of ex vivo tissue (left) and the corresponding 3D rendering of the ex vivo vasculature (right).

FIG. 5 shows an example of a NN training workflow according to an embodiment of the present disclosure.

FIG. 6 shows processing steps for training a convolutional NN according to an embodiment of the present disclosure.

FIG. 7 is a flow diagram demonstrating an overview of the acquisition of experimental labeled training data used to train the deep learning NN for SR-UMI.

FIG. 8 shows an example flowchart for leveraging CAM optical and acoustic imaging to provide a ground truth SR-UMI dataset for NN training according to an embodiment of the present disclosure.

FIG. 9 demonstrates a workflow used to generate a training dataset for the NN according to an embodiment of the present disclosure.

FIG. 10 illustrates an output and analysis unit according to an embodiment of the present disclosure.

FIG. 11 illustrates a signal processing chain from microbubble signal input to various types of output according to an embodiment of the present disclosure.

FIG. 12 shows an example of additional processing that may be performed to process the microbubble signal before sending into the NN according to an embodiment of the present disclosure.

FIG. 13 illustrates a method of using “scouting” microbubbles to adaptively train the deep learning NN based on a specific imaging subject (e.g., different patients) during imaging according to an embodiment of the present disclosure.

FIG. 14 shows examples of using Field-II to generate synthesized training data according to an embodiment of the present disclosure. (a) Field II simulation of the randomly distributed scatterers that emulate microbubbles in (c). (a) shows the intensity of the microbubble ultrasound signal (i.e., the envelope of the RF signal). (b) shows the RF signal of the microbubbles simulated by Field II.

FIG. 15 shows examples of localization results using Field-II simulation (intensity envelope and RF) trained network according to an embodiment of the present disclosure.

FIG. 16 is an example U-Net architecture for generating super-resolution localization maps.

FIG. 17 shows super-resolved vessel maps of the CAM surface vessels obtained by accumulating locations of microbubbles in 1600 frames (corresponding to 1.6 seconds), using envelope (ENV), RF, and the conventional methods according to embodiments of the present disclosure.

FIG. 18 shows an example of real-time display of blood flow in a mouse brain performed according to an embodiment of the present disclosure.

FIG. 19 shows an example spatial-temporal training using simulated MB flow data and neural network.

FIG. 20 shows an example LSTM architecture for super-resolution microbubble velocimetry

FIG. 21 shows an example simulation procedure of a MB flow model based on CAM optical image of CAM surface vessel. (a) shows the green channel of the optical image. (b) is the segmentation result based on (a). (c) (d) are the skeleton image and distance map obtained by medial-axis skeletonization. (e) is the graph representation of (c). (f) shows the assigned flow direction of the vessel network. (g) is the flow direction map and (h) is the flow velocity map of the structure.

FIG. 22 shows examples of super-resolved blood flow velocity maps of CAM surface vessel generated using DL-based ultrasound microbubble velocimetry (a) and conventional ULM (b), and (c) shows the trace of the SMV-measured average flow velocity of the vessels over a duration of 1.6 s.

FIG. 23 demonstrate a short data segment of microbubble data (a) that was processed using conventional ULM (b) which resulted in sparse flow velocity data (c). Direct accumulation of microbubble signal reveals diffraction-limited vessel structure (d). Processing the same data segment with DL-based ultrasound microbubble velocimetry allowed for super-resolution velocity estimation with more structural connectivity than conventional ULM (e). (f-i) show a detailed example of super-resolved blood flow velocity mapping of CAM surface vessels, with optical reference (f). DL-based ultrasound microbubble velocimetry (g) outperformed conventional ULM (h) for this longer duration dataset. DL-based ultrasound microbubble velocimetry shows distinct pulsatility features (i) in local regions sampled from feeding (Art) and draining (Vein) vasculature, which matched conventional Doppler velocity measurements.

FIG. 24 demonstrate a detailed flow dynamics analysis on CAM surface vessels using DL-based ultrasound microbubble velocimetry. (a) are video frames taken from a DL-based ultrasound microbubble velocimetry generated pulsatility video. Selected regions from an accumulated DL-based ultrasound microbubble velocimetry map (b) demonstrate a gradual decrease in peak velocity and pulsatility in velocity profiles (c, Region 1) along the length of the vessel (labelled i to iv). Region 2 demonstrates a phase delay in peak velocity estimates for vessels label Art and Vein.

FIG. 25 demonstrates the influence of different vessel structure priors on DL-based ultrasound microbubble velocimetry. (a) shows the sparse results of conventional ULM with a 1.6 second long dataset taken from a mouse brain, along with conventional ULM with a 64 second long accumulation (b). The short dataset was processed with DL-based ultrasound microbubble velocimetry trained with CAM simulation data (c), which demonstrates acceptable performance for larger subcortical vessels but is unable to separate parallel cortical vessels. Training the deep network with a mouse brain prior (d) improves performance in the cortical regions, while maintaining the ability to reconstruct subcortical features.

FIG. 26 demonstrates DL-based ultrasound microbubble velocimetry analysis of a mouse brain dataset. Velocity profiles (a) were generated for cortical and subcortical vasculature taken from a DL-based ultrasound microbubble velocimetry velocity map of the mouse brain (b). Velocity traces of cortical and subcortical vessel segmentations showed good correspondence to external validation with an electrocardiogram (c).

FIG. 27 is a block diagram of an example super-resolution ultrasound microvessel imaging and microbubble velocimetry system according to some embodiments of the present disclosure.

FIG. 28 is a block diagram of example components that can implement the system of FIG. 27.

### DETAILED DESCRIPTION

Described here are systems and methods for super-resolution ultrasound microvessel imaging and velocimetry. The systems and methods utilize deep learning and parallel computing to realize real-time super-resolution microvascular imaging and quantitative analysis and display.

In some embodiments, the systems and methods generate super-resolution microvessel images (e.g., vessel maps), which may be generated based on fast localization and tracking to construct a super-resolution image. Quantitative analysis of the localization or tracking result can also be generated. Examples of quantitative analysis can include, but are not limited to blood flow speed measurements, microbubble counting, morphometric analysis (e.g., vessel diameter, vessel spatial density, vessel structure, tortuosity, vessel length, and descriptions of vascular organization), and functional analysis (e.g., flow aberration, flow turbulence, characterization of velocity profiles, time-varying changes in blood flow and velocity, and pulsatility and pressure estimation).

In some other embodiments, the systems and method generate super-resolution velocimetry maps (e.g., velocity maps, which may include both speed and direction information). Advantageously, the velocimetry maps can be generated without needing conventional microbubble localization processing. Direct quantitative outputs can also be generated based on the velocimetry maps. Example outputs from the quantitative analysis can again include blood flow speed measurements, microbubble counting, morphometric analysis, and functional analysis.

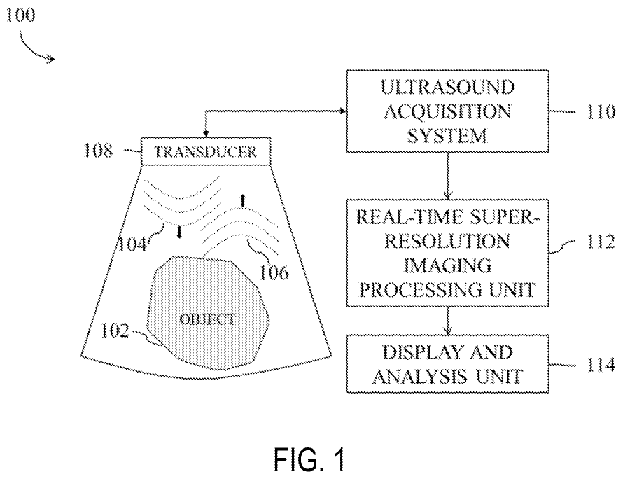

FIG. 1 illustrates an example super-resolution (“SR”) ultrasound microvessel imaging system 100. The SR ultrasound microvessel imaging system 100 include at least one ultrasound transducer 108 that transmits at least one ultrasound wave 104 into tissue 102. The ultrasound wave 104 interacts with the tissue 102 and results in another wave 106 that propagates towards the ultrasound transducer 108. Ultrasound signal data are acquired by an ultrasound acquisition system 110 and then processed by a real-time super-resolution imaging processing unit 112, which generates the results (e.g., microvessel images) that can be displayed and analyzed by the display and analysis unit 114. The ultrasound transducer 108 can include single element transducers; one-dimensional ultrasound arrays; 1.5-dimensional ultrasound arrays; two-dimensional ultrasound arrays; mechanical-translation-based three-dimensional imaging arrays (also known as the wobbler or the sweeper arrays); wearable ultrasound transducers; catheter ultrasound transducers: transducers with arbitrary shapes, such as a linear array, a curved array, a ring array, an annular array, a phased array, and a hockey stick array; and other such ultrasound transducer configurations.

The ultrasound data being acquired and processed by the ultrasound acquisition system 110 can include radio frequency (“RF”) ultrasound signal data and/or in-phase quadrature (“IQ”) ultrasound signal data. The real-time super-resolution imaging processing 112 can be integrated with the ultrasound acquisition system 110, can be a stand-alone device that interfaces with the ultrasound acquisition system 110 for data processing, or can be a cloud-based service that communicates with the ultrasound acquisition system 110 for data processing.

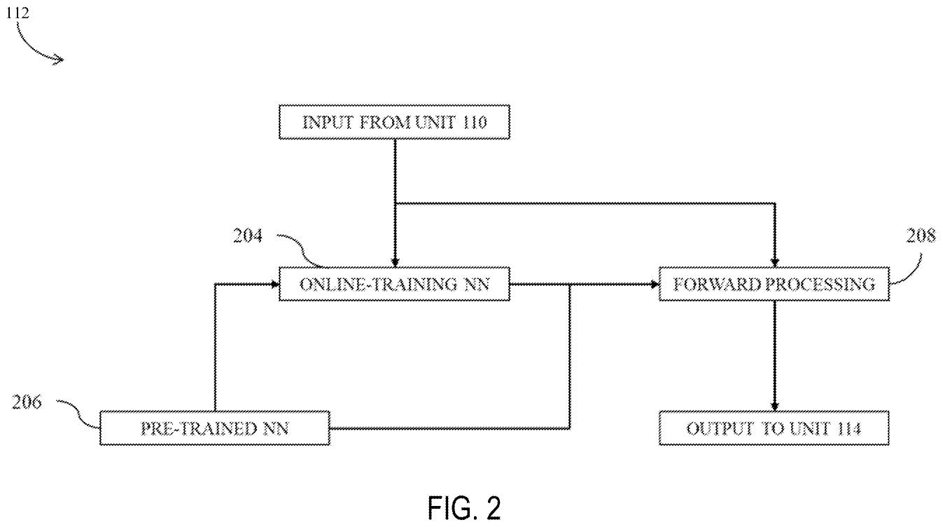

The real-time super-resolution imaging processing unit 112 can include several components, such as the example components illustrated in FIG. 2. For example, the components illustrated in FIG. 2 can be implemented.

With reference now to FIG. 2, the real-time super-resolution imaging processing unit 112 can include an online training neural network (“NN”) component 204, a pre-trained neural network (“NN”) component 206, and a forward processing unit 208. These and other components of the real-time super-resolution imaging processing unit 112 can be implemented as hardware, software, a combination of hardware and software, software in execution, or the like.

The pre-trained NN component 206 can implement methods of training a NN that can process the ultrasound signal data from the ultrasound acquisition unit 110 via the forward processing unit 208. The online-training NN can implement methods of adaptively training a NN based on data that is being collected at the same time during ultrasound imaging from the ultrasound acquisition unit 110. The trained NN is then used by the forward processing unit 208 to process the input (e.g., ultrasound signal data) from the ultrasound acquisition unit 110. The forward processing unit 208 can implement methods of using either the pre-trained NN 206 or the online-training NN 204 to process the input data from the ultrasound acquisition unit 110. The output generated by the forward processing unit 208 is then sent to the display and analysis unit 114 for further processing, analysis, and/or display.

The pre-trained NN component 206 of the real-time super-resolution image processing system 112 can implement methods and processing steps of constructing a NN that can process the input ultrasound signal data into different formats that can be used for super-resolution ultrasound microvessel imaging and/or super-resolution microbubble velocimetry.

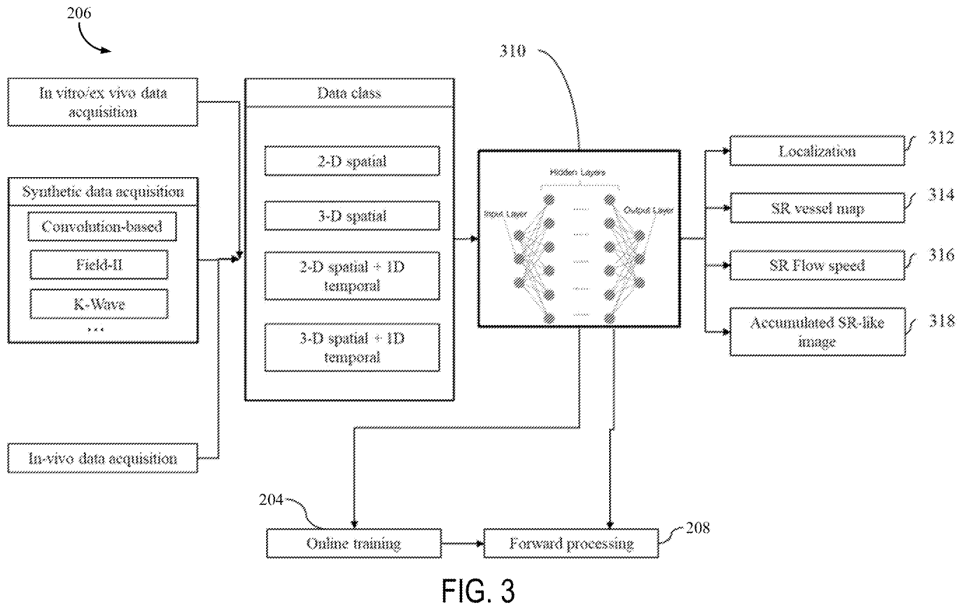

As shown in FIG. 3, the NN can be trained by at least one of synthetic data, in vitro data, ex vivo data, in vivo data, and/or the combination of different types of data.

Synthetic data can be obtained by computer simulation of microbubble signals observed under ultrasound. For example, synthetic data can be based on a direct convolution between microbubble location and the point-spread-function (“PSF”) or impulse response (“IR”) of the ultrasound system, ultrasound simulation software Field II, K-wave simulation data, and/or microbubble signals generated by using a generative neural network (e.g., a generative adversarial network (“GAN”)). The known microbubble locations, motions, and signals used in the simulation can be used as the ground truth or reference for training the NN 310. The synthetic data can include randomly distributed point scatterers that behave similarly to contrast microbubbles. Microvasculature can be simulated with assigned flow speed and vessel geometry for distributing the point scatterers, which will be displaced by the assigned local flow speed at each time instant. The microbubble dimension and shape can be spatially and temporally varying.

In vitro data can be obtained by using a tissue-mimicking phantom or point targets to generate ultrasound data that can be used for training the NN 310. As a non-limiting example, a flow phantom can be used to mimic blood flow in a vessel and perfused with microbubbles to generate ultrasound signal data for training. The microbubble concentration can be carefully controlled so that individual microbubble signals can be identified either manually or computationally by image processing. The manually and/or computationally identified microbubble signals and/or microbubble locations can be used as ground truth for training the NN 310. As another non-limiting example, experimental microbubble signals can be obtained by acquiring microbubble data from liquid solutions, other samples, or in vivo. Experimental microbubble data can then be directly used as PSFs (e.g., by directly using the image of a single microbubble as a PSF), or can be processed to generate PSFs (e.g., by model fitting with a Gaussian function, or the like). As another non-limiting example, the in vitro data can be obtained by ultrasound imaging of a point target (e.g., a glass sphere) submerged in water. By moving the target to different spatial locations, one can sample spatially-varying signals that represent microbubbles detected from different spatial locations. The movement of the target can also be controlled to generate temporally-varying signals that represent microbubble velocities.

Ex vivo data can be obtained by ultrasound imaging of harvested biological tissue with contrast microbubbles. As a non-limiting example, a swine liver can be harvested and re-perfused with blood-mimicking fluid and microbubbles to obtain ultrasound data. Similar to the example in vitro data acquisition described above, when acquiring ex vivo data the microbubble concentration can be carefully controlled so that individual microbubble signals can be identified either manually and/or computationally by image processing. The manually and/or computationally identified microbubble signals and/or microbubble locations can then be used as ground truth for training the NN 310.

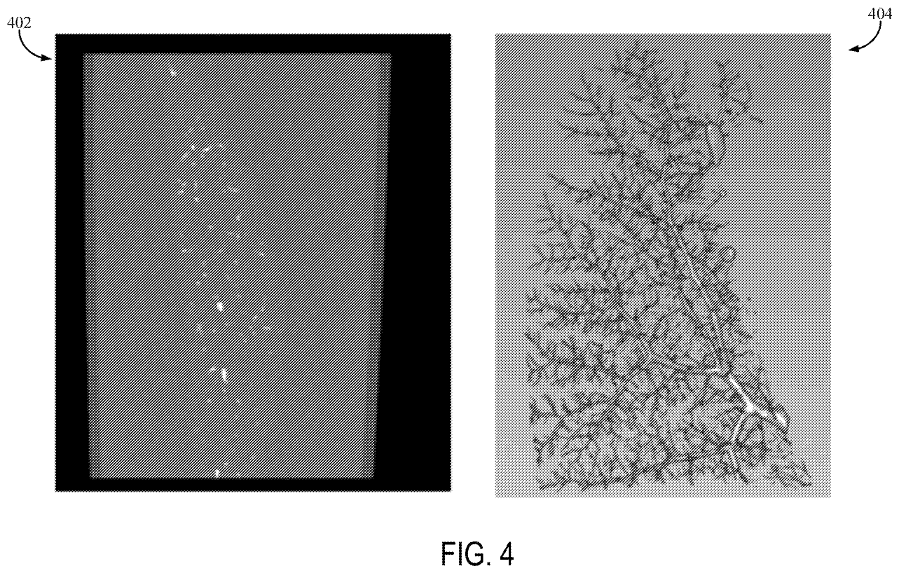

Ex vivo data can also be used to inform the synthetic data acquisition by providing vascular structure and geometry for the distribution of point scatterers and for the assignment of local flow speeds. As a non-limiting example, ex vivo tissues can be perfused with a microCT contrast agent that solidifies in place (e.g., Microfil) to provide three-dimensional imaging volumes of patent vasculature. An example of this is illustrated in FIG. 4. In the illustrated embodiment, a rabbit liver was perfused with an X-ray contrast agent and imaged using a micro-CT system (image 402). Other non-limiting examples include any vascularized structure (e.g., kidney, brain, spleen, and muscle) extracted from any multicellular organism. The vascular structure in this ex vivo tissue was extracted via thresholding and seeded region growing and is demonstrated as an iso-surface rendering 404. These forms of image reconstruction, analysis, and segmentation are for demonstrative purposes only and do not represent an exhaustive list of applicable techniques. This vascular geometry can then be used to generate synthetic training data using any of the techniques described in the present disclosure.

In some other embodiments, the formation of synthetic data can be informed by data acquired (e.g., ex vivo, in vitro, or in vivo data) using other imaging modalities in order to provide vascular structure and geometry for the distribution of point scatterers and for the assignment of local flow speeds. Other non-limiting examples for acquiring data that can inform synthetic data formation include microCT with other contrast agents (e.g., iodine-based contrast agents, gadolinium-based contrast agents), contrast-enhanced magnetic resonance imaging, white-light optical imaging, wide-field fluorescence, confocal microscopy, 2-photon microscopy, multi-photon microscopy, histological sectioning (e.g., immunostaining, perfusion of dyes and fluorescent markers, colorimetry, serial sectioning, cryosectioning, and en face pathological assessment of tissue structure(s) and vascular components), optical coherence tomography, positron emission tomography, single photon emission tomography, and any combination of the aforementioned imaging modalities and techniques.

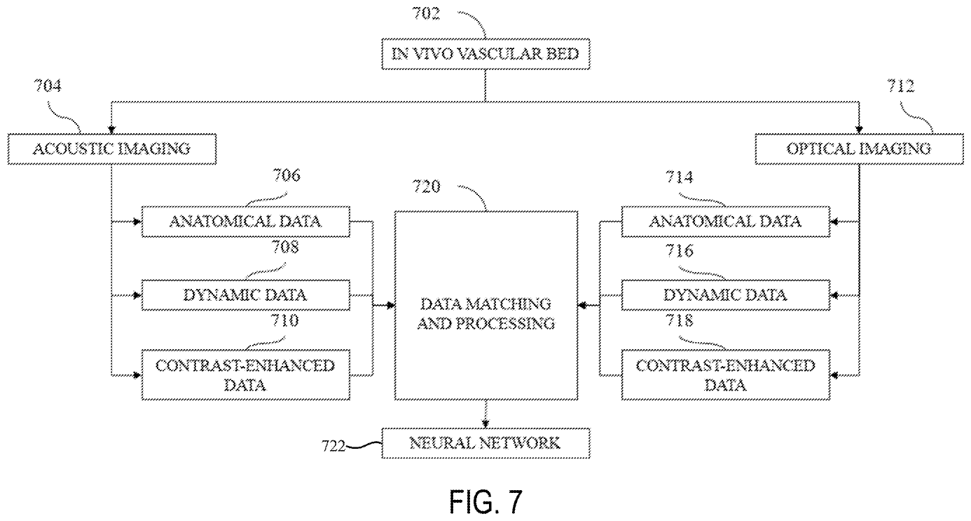

In vivo data can be obtained by any one of various methods, such as those described below in more detail. In general, these methods can include ultrasound imaging of in vivo tissue such as animals, chicken-embryos, or humans, and the concurrent optical and ultrasound imaging of in vivo tissue such as chicken-embryos. For the ultrasound-only approach, a similar method of obtaining microbubble signals as in the in vitro and ex vivo data acquisition example described above can be used. The microbubble concentration can be carefully controlled so that individual microbubble signals can be identified either manually and/or computationally by image processing. The manually and/or computationally identified microbubble signals and/or microbubble locations can then be used as ground truth for training the NN 310. The concurrent optical and ultrasound imaging method is described in more detail below (e.g., with respect to FIGS. 7-9).

Similar to the above-mentioned examples with ex vivo data, in vivo data acquisition can also be used to inform the formation of synthetic data by providing vascular structure and geometry for the distribution of point scatterers and for the assignment of local flow speeds via direct blood velocity measurement, microbubble trajectory measurement, or via numerical simulation. This example of data management does not necessarily require concurrent ultrasound acquisition. The non-limiting case examples of imaging modalities described above with respect to acquiring ex vivo data to inform formation of synthetic data can also be used to acquire in vivo data. A specific non-limiting example includes optical imaging of a superficial vascular bed (e.g., chorioallantoic membrane or retinal fundus imaging) to provide a physiologically relevant vascular structure for flow simulation.

The input to the NN 310, for both training and forward processing (e.g., forward-propagation) purposes, includes at least one of beamformed or unbeamformed ultrasound signal data, compounded or non-compounded RF ultrasound signal data, IQ ultrasound signal data, envelope of RF, magnitude of IQ, real part of IQ, and imaginary part of IQ. The dimension of the data can be two-dimensional in space, three-dimensional in space, two-dimensional in space and one dimensional in time, and three-dimensional in space and one dimensional in time. The input to the NN 310 can be one or a combination of different types of data formats.

For training input data that only have spatial information, the ground truth can include the true locations of the microbubbles or the images of microbubbles that reflect the true dimension and location of the microbubbles. The underlying tissue microvasculature can also be used as ground truth for training. Using backpropagation, the network is trained to recover these locations and/or microbubble images from the ultrasound input.

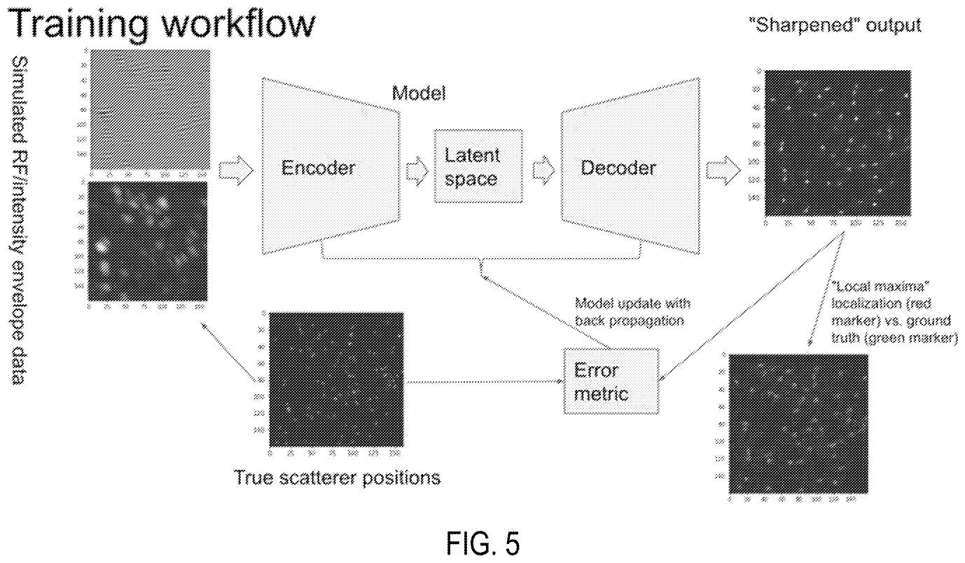

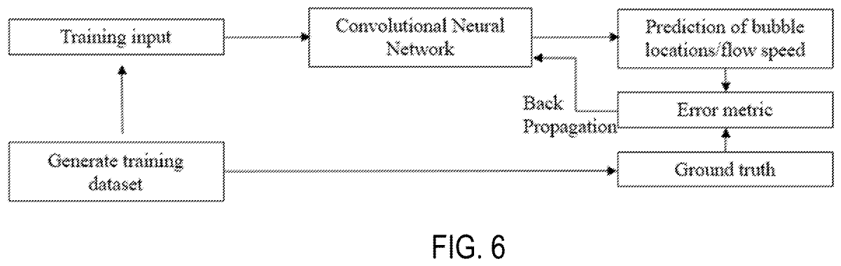

FIG. 5 shows an example of the NN training workflow, and FIG. 6 shows the general processing steps and methods for training a convolutional NN as a specific example. Either simulated ultrasound IQ data or RF data can be used as the input to the NN. The ground truth from the simulation can be used to derive error metrics for training. The output from the NN can be one of, or a combination of, sharpened microbubble images that can be directly used for imaging purposes or subject to further super-resolution processing (e.g., localization and tracking), or a super-resolved velocimetry map. For spatial-temporal input data, two different types of ground truth can be used: true microbubble locations and/or microbubble images that reflect the true dimension and location of the microbubble at different time instants, and local blood flow velocity (both speed and direction) at each individual microvessel. Instead of training based on only the spatial location of individual microbubbles, the network can be optimized to recover the microvascular flow speed based on spatiotemporal microbubble data. For the purposes of clarity in this disclosure, a NN that is trained to estimate the local blood flow velocity of microvessels without an explicit microbubble localization step is referred to as SMV (“super-resolution microvessel velocimetry”), which can be considered as a subset of NN-based super-resolution ultrasound microvessel imaging.

Referring again to FIG. 3, the output from the NN 310 can include different types of data that include at least one of the locations of the microbubbles (block 312), super-resolved microvessel maps (block 314), super-resolved microvessel flow speed map (block 316), and/or accumulation of super-resolution-like images (block 318). The NN 310 is trained separately based on the different types of output data. The trained NN can be used either by the online training unit 204 of the real-time super-resolution imaging processing unit 112 or the forward processing unit 208 of the real-time super-resolution imaging processing unit 112. Note that as shown in FIG. 3, many possible combinations of input data type, input data class, and output data type can be used in the pre-trained NN unit 206. Each possible combination can be implemented by a specific NN that is trained with a specific combination of the input and output data.

FIGS. 7-9 illustrate example methods of in vivo data acquisition that can be used to provide training data for training the NN 310 in the pre-trained NN unit 206 of the real-time super-resolution imaging processing unit 112. In some embodiments, deep learning NN training schemes based on experimental optical imaging and ultrasound imaging data are used to develop a NN for real-time super-resolution ultrasound microvessel image processing. At present, few attempts have been made to implement deep learning for super-resolution ultrasound microvessel imaging. A primary reason is the difficulty in obtaining realistic labeled data for training, which is a common issue with deep learning applications in medical image reconstructions in general. As a result, simulated or synthesized data are typically used for training, which is not ideal for developing a robust NN.

The systems and methods described in the present disclosure overcome these drawbacks by using in vivo vascular bed models (e.g., based on chicken embryo-based microvessel imaging) for deep learning NN training. For example, the chorioallantoic membrane (“CAM”) offers a unique setup for deep learning NN training because the CAM microvessels can be imaged simultaneously by ultrasound and optical microscopy. Because optical imaging provides adequate spatial resolution to resolve microvessels as small as capillaries, and because it can also image individual microbubbles, it can serve as the ground truth for reliable deep learning NN training. In some embodiments, training data can additionally or alternatively include non-contrast-enhanced ultrasound signal data. That is, the neural network can be trained on and applied to non-microbubble data. For example, chicken embryo red blood cells are very strong acoustic scatterers, so non-contrast blood flow images can be generated without using microbubble contrast agents in some instances.

As another example, the systems and methods described in the present disclosure can use other types of vessel data for training deep learning models and neural networks. For instance, in vivo and/or ex vivo animal brain vasculature can be imaged and used as a training data set. In some example studies, in vivo blood flow in a mouse brain can be imaged using ultrasound or other imaging techniques and used as a training data set.

FIG. 7 illustrates an overview of a process for acquiring experimental labeled training data that can be used to train the deep learning NN for super-resolution ultrasound microvessel imaging. Experimental data can be obtained from an in vivo vascular bed (step 702), which can include—but is not limited to—capillaries, venules, arterioles, and larger vascular components which contain, as non-limiting examples, freely flowing erythrocytes, lymphocytes, blood plasma, and/or lymphatic fluids. This vascular bed may also have naturally occurring or induced vascular pathologies such as flow occlusions, gas embolism, lipid embolism, thrombosis, stenosis, sclerosis, foreign body blockage, laceration, leakage, and/or other wounding. In principle, in vitro microfluidic devices and/or tissue mimicking flow phantoms can also be used to generate an optical/acoustic training dataset for the NN.

These vascular beds can also be enhanced with ultrasound and/or optical contrast agents, which can be injected into the intravascular space, stromal space, or other bodily tissues/fluids; or, these contrast agents can be added topically to allow for diffusion into the tissue. A non-limiting example of acoustic contrast agents are microbubbles, which generate ultrasound contrast due to a high acoustic impedance mismatch with tissue and/or via a non-linear acoustic response. These microbubbles can contain a gas-filled core (some examples include, but are not limited to, perfluorocarbon gasses, oxygen, atmospheric gasses, and/or other neutral gasses) or can contain a solid core (such as micro/nano-spheres). These microbubbles may be introduced into the vascular bed, or may be spontaneously/naturally occurring, such as in transient cavitation/nucleation. These microbubbles can be stabilized with outer shells of lipids, proteins, carbohydrates, phospholipids, any combination of the above, or can be shell-less. Typically, microbubbles are sized small enough to freely pass through the capillary lumen when intravascularly injected; however, poly-disperse and/or size-selected microbubble populations may include larger or smaller microbubble diameters. Generally, microbubbles are limited to the blood pool when introduced into the intravascular space and will not extravasate. The microbubble shell can be modified to include antigens (for targeted imaging of protein expression, such as VEGF-R to target angiogenesis), fluorescent indicators, biotin/avidin/streptavidin binding sites, or can incorporate modified lipids, proteins, or carbohydrates. The microbubbles can be labelled for optical imaging with fluorescent indicators (either via binding to the aforementioned biotin/avidin/streptavidin binding sites or via incorporation into the core or shell components), quantum dots, or optical dyes; or, the microbubbles can be modified to increase optical scattering or increase optical contrast with tissue.

Other non-limiting examples of acoustic contrast agents include nanobubbles and nanodroplets, which may freely extravasate to permit extravascular super-resolution ultrasound microvessel imaging. These contrast agents can be labelled and targeted in a similar manner to the above-mentioned microbubbles. Phase-change nanodroplets can condense and vaporize in response to acoustic energy, allowing for a broader spectrum of NN super-resolution ultrasound microvessel imaging training data. The tissues of the vascular bed can also be labelled to provide acoustic and/or optical contrast, such as fluorescent labelled of the endothelial lumen.

An in vivo vascular bed imaged for NN training is preferably accessible to both optical and acoustic imaging. For example, the CAM microvasculature (described above) provides an advantageous experimental model for NN training because it is low cost (allowing for the generation of a large training dataset for robust learning), has minimal tissue motion and signal attenuation, and is easy to manipulate. Surgical intervention can be used to give optical and acoustical access to other vascular networks, such as exposed muscle, mammalian placenta, cranial window, or skin. Vascularized hollow organs, such as retinal fundus imaging, could also provide an appropriate in vivo vascular bed for NN training.

Once the experimental set-up has been adequately prepared, the vascular bed undergoes acoustic imaging (step 704). Acoustic imaging can include, but is not limited to, any suitable form of ultrasound signal acquisition and image reconstruction. Non-limiting examples can include ultrasound imaging in the high-frequency range (e.g., greater than 20 MHz), the clinical frequency range, or the low frequency range. Acoustic imaging equipment can include, as non-limiting examples, single element transducers, linear transducers, curvilinear transducers, phased-array transducers, matrix-array or 2D transducers, row-column array transducers, capacitive micro-machined ultrasound transducers (“CMUTs”), transmitting in line-by-line, plane wave, synthetic aperture, continuous wave, ensemble, or manual motion acquisitions. Ultrasound imaging modes include, but are not limited to, fundamental imaging, harmonic imaging, sub-harmonic imaging, super-harmonic imaging, contrast imaging, Doppler imaging, non-linear imaging, or amplitude-, phase-, or frequency-modulated imaging.

The received ultrasound signal data, which may include element or channel RF data, image RF data, IQ data, RAW data, log-compressed data, or streaming video data, are then analyzed to extract anatomical information (step 706), dynamic information (step 708), and/or contrast-enhanced data (step 710) from the vascular bed. Some non-limiting examples of anatomical information (determined at step 706) include the location and dimensions of the vascular lumen, the location and dimensions of surrounding tissues and stromal space, or other morphological or textural features. Some non-limiting examples of dynamic information (determined at step 708) include the movement of red blood cells, tissue motion, transducer motion, flow pulsatility, blood flow and fluid flow velocity profiles, tissue phase aberration, shear wave propagation, tissue deformation, and imaging acquisition noise. Contrast-enhanced data (determined at step 710) can include the location and motion of intravascular contrast microbubbles, non-linear contrast agent acoustical response, the location and motion of extravascular nanodroplets, the phase-change (e.g., condensation/vaporization) of nanodroplets, the binding kinetics of targeted contrast agents, microbubble disruption, inflow of contrast agent, and the perfusion kinetics of contrast agents.

In addition to acoustical imaging, the vascular bed is also imaged using optical imaging (step 712), which generates analogous anatomical data (step 714), dynamic data (step 716), and contrast-enhanced data (step 718) for analysis. Optical imaging can include, but is not limited to, both upright and inverted microscopy, conventional photography, white-light microscopy, fluorescent microscopy, confocal microscopy, two-photon and multi-photon microscopy, Raman microscopy, scanning microscopy, transmission microscopy, x-ray microscopy, and electron microscopy. Optical images can be generated either via diffraction limited imaging or by super-resolution optical techniques such as the following non-limiting examples, structured illumination microscopy, spatially modulated illumination microscopy, optical fluctuation imaging, stochastic optical reconstruction microscopy, and photo-activated localization microscopy.

Non-limiting examples of the anatomical information (determined at step 714) that can be generated via optical imaging include the position and size of vascular lumen, the position and size of surrounding tissues, the location, binding efficiency, and intensity of fluorescent indicators bound to various tissues and protein targets, the structural composition and organization of tissues, and the abundance of extravasated indicators. Dynamic information available to optical imaging (determined at step 716) can include but is not limited to, tissue motion, the movement of red blood cells, flow pulsatility, blood flow and fluid flow velocity profiles, tissue deformation, photobleaching of indicators, the activation/excitation/emission of fluorescence, changes to illumination (white-light, single wave-length, multi-wavelength electromagnetic waves), structured illumination, and laser scanning. Contrast-enhanced data (determined at step 718) can include the position and movement of optically-labeled and unlabeled microbubbles, the position and movement of optically-labeled and unlabeled nanodroplets, the position and movement of optical dyes, fluorescent indicators, protein expression markers, and the position and movement of freely flowing and fixed fluorescent particles.

The acoustic imaging data acquisition and the optical imaging data acquisition are synchronized both in space and in time to capture matched biological signals. A non-limiting example of the synchronization is achieved by triggering the optical imaging acquisition with the acoustic imaging device, or by triggering the acoustic imaging acquisition with the optical imaging device, or by retrospectively aligning the data. The anatomical, dynamic, and contrast-enhanced data from both the acoustic imaging and the optical imaging are then sent to data matching and processing (step 720). The purpose of this step is to generate training datasets to be fed into the neural network (at step 722). Some non-limiting examples of data matching include the pairing and tracking of ultrasound contrast microbubbles, which have been optically labelled to serve as a ground truth for super-resolution ultrasound microvessel imaging localization. Other non-limiting examples might include the comparison of microbubble localization trajectories to optically determined vascular lumen, the confirmation of microbubble velocities between the two modalities, or the validation of microbubble count processing.

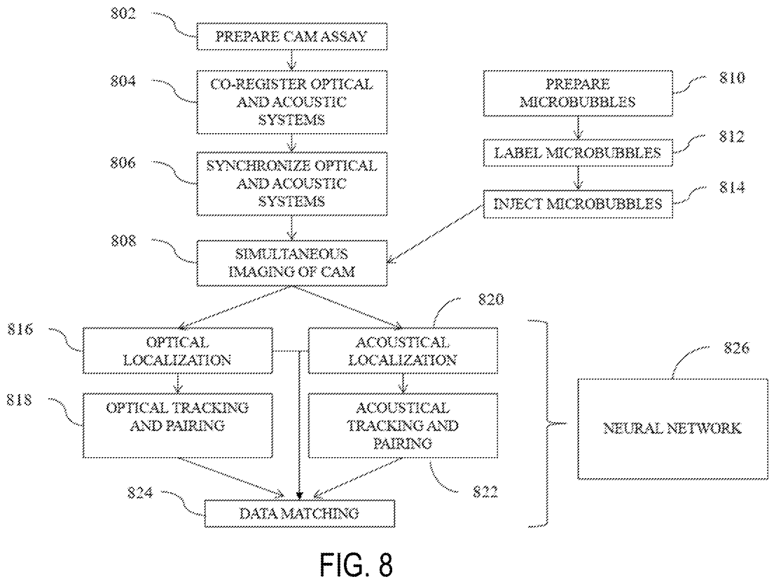

FIG. 8 shows one example of a process for leveraging CAM optical and acoustic imaging to provide a ground truth super-resolution ultrasound microvessel imaging dataset for NN training. A chorioallantoic membrane assay can be generated either in ovo (in the shell) or ex ovo (out of the shell) from commercially available fertilized eggs in advance of imaging (step 802). An ex ovo assay may be preferred to facilitate optical imaging. The species of fertilized egg can be selected as the most appropriate based on several criteria (such as cost, commercial availability, incubation time, complexity and development time of vasculature, and/or availability of labeling antibodies), and can include, but is not limited to, avian eggs, reptile eggs, or amphibian eggs.

The optical and acoustic systems are spatially co-registered (step 804) to provide the same imaging information. Some non-limiting examples of co-registration include fiducial localization, intensity-based alignment, feature-based alignment, spatial-domain alignment, frequency-domain alignment, interactive registration, automatic registration, cross-correlation, mutual information, sum of squares differences, ratio of image uniformity, curve matching, surface matching, or physical registration. In some embodiments, the acoustic and/or optical data are transformed before co-registering their multi-modality imaging information. Some non-limiting examples of transformations can include translation, shearing, rotation, affine transformations, rigid transformations, non-rigid deformations, scaling, and cropping.

After being spatially registered, the acoustic and optical data acquisitions are synchronized to provide temporal registration (step 806). This step ensures that tracking applications are using the same target for NN training. A non-limiting example of synchronization can include using either an external or internal trigger in/out signal to ensure that the acoustic and optical system(s) are acquiring data at the same point in time. Some other non-limiting examples would be to use internal time-stamps, to temporally interpolate datasets, to use mutual information to temporally register, or to rely on co-incident events.

Once the acoustic and optical systems have been adequate registered spatially (at step 804) and temporally (at step 806), simultaneous imaging of the CAM can commence (step 808). This unit is responsible for generating the majority of the experimental optical imaging data to develop and train deep learning NNs super-resolution ultrasound microvessel imaging. Prior to, or in parallel to, this step, the microbubble contrast agents (or equivalent) are prepared (step 810). These can include, but are not limited to, the acoustical contrast agents that were discussed above. In some embodiments, these microbubbles may require labelling for optical imaging (step 812), although this step may be skipped depending on the specifics of the optical system and mechanism(s) of optical contrast. A non-limiting example, as discussed above, would be to fluorescently tag the outer membrane of the microbubbles for fluorescent microscopy. These microbubbles are then added to the CAM assay, such as via intravascular injection (step 814) or other suitable method for administering the microbubbles to the CAM assay.

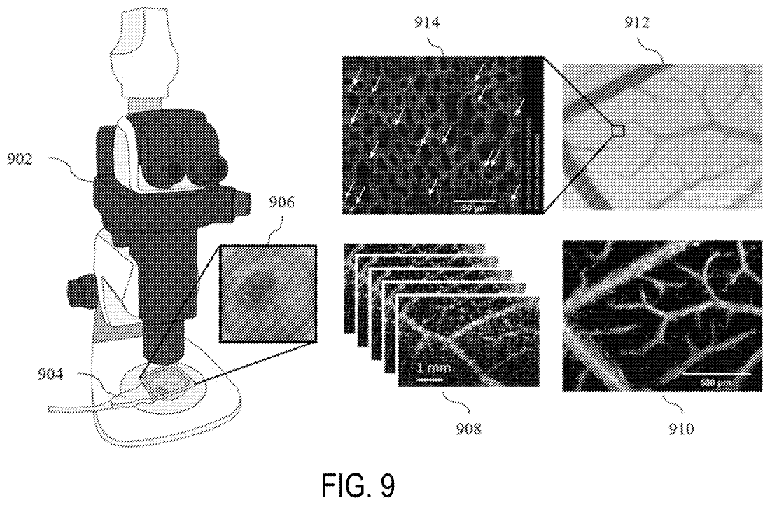

From step 808, optical and acoustic microbubble events are detected, recorded, and tracked. A more in-depth description of microbubble signal processing is described below with respect to FIG. 9.

FIG. 9 illustrates a non-limiting example of a workflow used to generate a training dataset for the NN. An optical imaging system 902 is co-registered with an acoustical imaging system 904 while imaging an ex ovo chicken embryo CAM assay 906. The ultrasound imaging data can be acquired as a three-dimensional matrix (lateral, axial, and temporal dimensions) and spatiotemporally filtered to generate microbubble signal data 908. These microbubble events are localized and tracked to serve as input into the NN, with an example super-resolution ultrasound microvessel imaging result demonstrated in image 910. The co-registered wide-field optical imaging serves as one example of a ground truth dataset to compare against (shown in image 912). Prior to imaging, this CAM can be injected with green-fluorescent microbubbles and a red-fluorescent vascular label. Confocal microscopy provides high-resolution imaging of localized microbubble events (shown in image 914) to serve as another type of gold-standard to train the NN's localization accuracy.

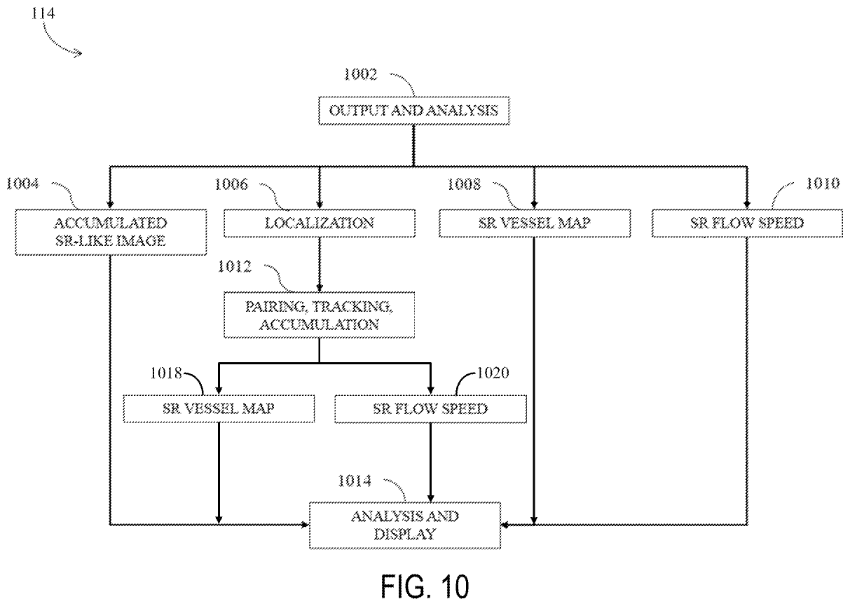

An example of a display and analysis unit 114 is further illustrated in FIG. 10. Depending on the different types of output data from the real-time super-resolution imaging processing unit 112, the display and analysis unit 114 uses different methods to process the signal. For example, if the output data includes sharpened microbubble image data, then process block 1004 is executed to directly accumulate the sharpened microbubble signals to achieve real-time display of tissue microvasculature, as indicated as output block 1014. If the locations of the microbubbles are being generated by the NN (process block 1006), then process block 1012 is executed to pair, track, and accumulate the microbubble signal to generate super-resolution vessel maps (process block 1018) and super-resolution flow speed maps (process block 1020). The corresponding results can be displayed and further analyzed to generate quantitative results, such as the number of microbubbles being detected (which is an indication of blood flow volume), the microvessel density, microvessel tortuosity, microvessel blood flow speed and flow direction, microvessel inter-vessel distance, microvessel oxygenation, microvessel blood flow turbulence, tissue flow rate, and tissue volume rate. In the specific case of SMV processing, the NN bypasses the explicit microbubble localization steps to directly output a super-resolved vessel map (process block 1008) and velocity map (process block 1010), which can then be sent for display and analysis at process block 1014.

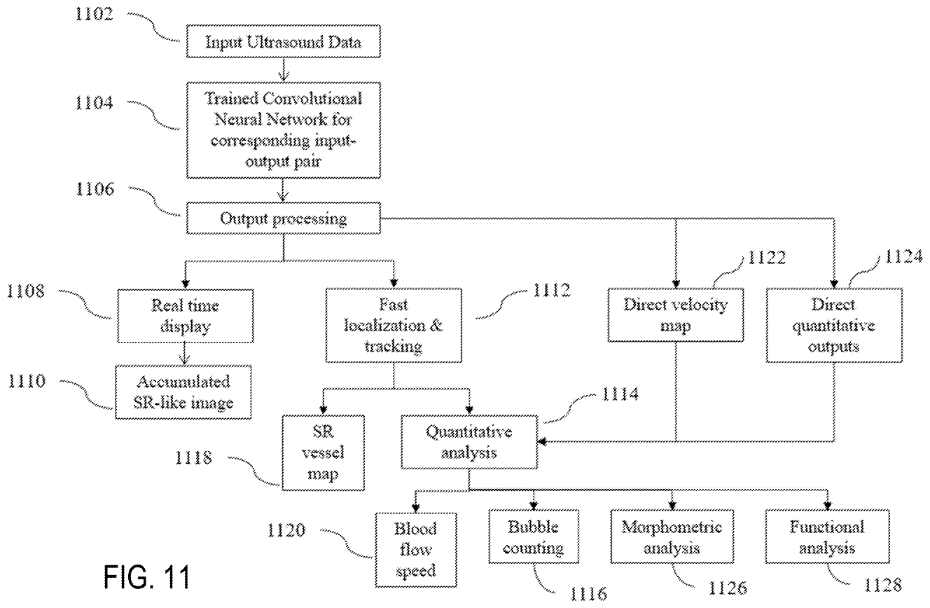

FIG. 11 illustrates an example signal processing chain from microbubble signal input to various types of output. The example uses a neural network (process block 1104) trained by a process described above for the desired input-output pair. The training can be fine-tuned based on the ultrasound imaging system used in practice. Acquired ultrasound signal data are input at process block 1102 and fed into the model. Process block 1106 is a fast post-processing process block that can convert the model output to various display formats.

The output can be directly displayed in real-time (process block 1108) or accumulated for a super-resolution-like vessel map (process block 1110). Otherwise, the super-resolution ultrasound microvessel imaging fast localization and tracking method (process block 1112) is applied to construct a super-resolution image (process block 1118). Process block 1114 performs quantitative analysis of the localization or tracking result, outputting various display results mentioned above. Examples of quantitative analysis can include, but are not limited to blood flow speed measurement (process block 1120), MB counting (process block 1116), morphometric analysis (process block 1126; non-limiting examples include vessel diameter, vessel spatial density, vessel structure, tortuosity, vessel length, and descriptions of vascular organization) and functional analysis (process block 1128; non-limiting examples include flow aberration, flow turbulence, characterization of velocity profiles, time-varying changes in blood flow and velocity, and pulsatility and pressure estimation).

As another non-limiting example, the microbubble signal input can follow the SMV network processing chain, where the input-output pair does not rely on conventional microbubble localization processing to produce super-resolved velocimetry maps (process block 1122) and direct quantitative outputs (process block 1124). These outputs can be fed into the same, or similar, downstream processing steps as above to generate accumulated SR-like images (process block 1110) and further quantitative analysis (process block 1114).

As described above, the systems and methods described in the present disclosure can utilize deep learning-based techniques to generate various different output data types for super-resolution ultrasound microvessel imaging and/or velocimetry. For example, a neural network can be trained to generate output as microbubble location data (e.g., block 312, block 1112), super-resolution microvessel images (e.g., block 314, block 1118), super-resolution velocimetry maps (e.g., block 316, block 1122), and/or accumulation of super-resolution-like images (e.g., block 318, block 1110). In some embodiments, a neural network is trained to generate one particular output data type (e.g., a microvessel image or a velocimetry map). In some other embodiments, a single neural network can be trained to generate two or more output data types (e.g., by having a different output node corresponding to each different output data type), for example, both a microvessel image and a velocimetry map.

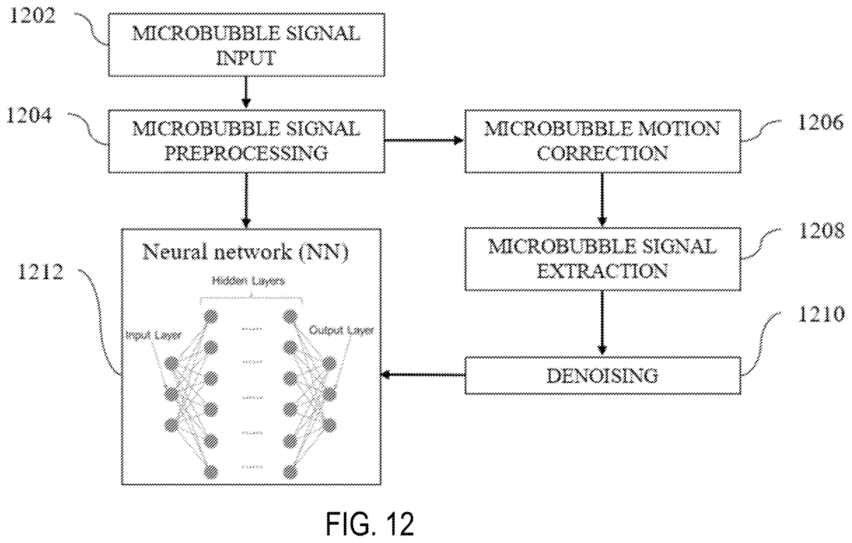

FIG. 12 shows an example of additional processing that may be implemented to process the microbubble signal before sending into the NN. These steps include motion correction (step 1206), microbubble signal extraction (step 1208) such as singular value decomposition (SVD)-based filtering, and denoising (step 1210) such as non-local means filtering.

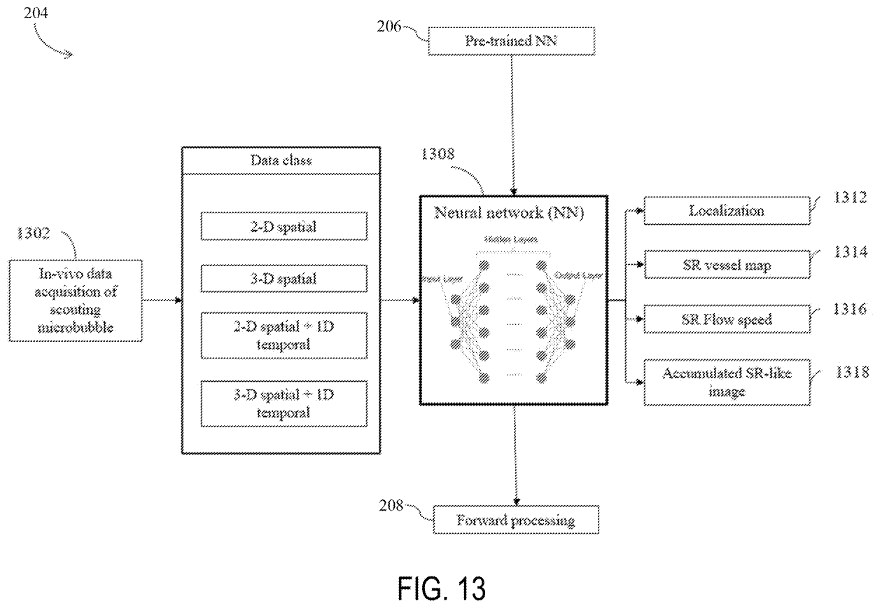

FIG. 13 illustrates a method of using “scouting” microbubbles to adaptively train the deep learning NN based on a specific imaging subject (e.g., individual patients) during imaging. For example, the illustrated embodiment is an example implementation on an online neural network training component 204 or a real-time super-resolution microvessel imaging processing unit 112.

Process block 1302 represents a step that involves injections of low concentration microbubbles (e.g., scouting microbubbles) into the subject to collect spatially sparse microbubble signals. The reason for the low concentration is to ensure that microbubble signals are not spatially overlapped so that individual microbubble signals can be conveniently recognized. Either manual or computational identification of the scouting microbubble signal can be used as the ground truth for the NN, which has been pre-trained using one of the methods introduced above. The scouting microbubble signal is used to fine-tune the NN so that it becomes adaptive to different imaging subjects. Because of different body habitus, applications (e.g. organs being examined), and imaging settings, the NN may not be optimally trained if using only the pre-trained NN. The scouting microbubble signal can be used to correct for subject-dependent acoustic phase aberration, attenuation, and other system-dependent variances such as different imaging sequences, frequencies, gains, and transducers. The online training can be done concurrently with imaging: as more and more scouting microbubble signals are being detected, the NN 1308 is being re-trained and fine-tuned simultaneously. The final online trained NN 1308 is then used for the rest of the imaging session on the same subject.

Like the neural network 310 trained using a pre-trained NN component 206, the output from the neural network 1308 trained with the online NN training component 204 can include different types of data that include at least one of the locations of the microbubbles (block 1312), super-resolved microvessel maps (block 1314), super-resolved microvessel flow speed map (block 1316), and/or accumulation of super-resolution-like images (block 1318).

The NN mentioned in this disclosure can be in any form. For spatial data, U-Net style convolutional neural network can be used to learn the internal patterns of microbubble behavior in spatial domain. For spatial-temporal data containing information of sequential frames, recurrent neural network can be leveraged to learn from bubbles' temporal behavior. Other types of NN or deep learning techniques such as generative adversarial network (“GAN”) and reinforcement learning can also be used. A specific, but not limiting, example of SMV using a long short-term memory neural network (“LSTM”) for spatial-temporal data training is detailed later in this disclosure.

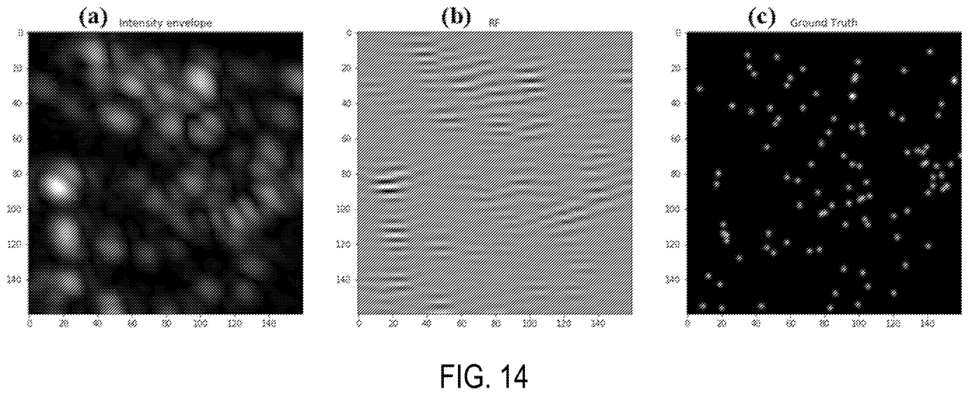

FIG. 14 shows examples of using Field-II to generate synthesized training data: Field-II synthesized training samples were generated in this example by simulating point scatterers randomly located in a 1 mm-by-1 mm area (e.g., FIG. 14 (c)). The number of scatterers per sample follows a random normal distribution within the range of 1-100 bubbles. The back-scattering intensities are assigned randomly to imitate the varying intensity as sources move in and out of the imaging plane. The region being imaged varies between different sampling frames to account for the spatially varying PSF of an imaging system.

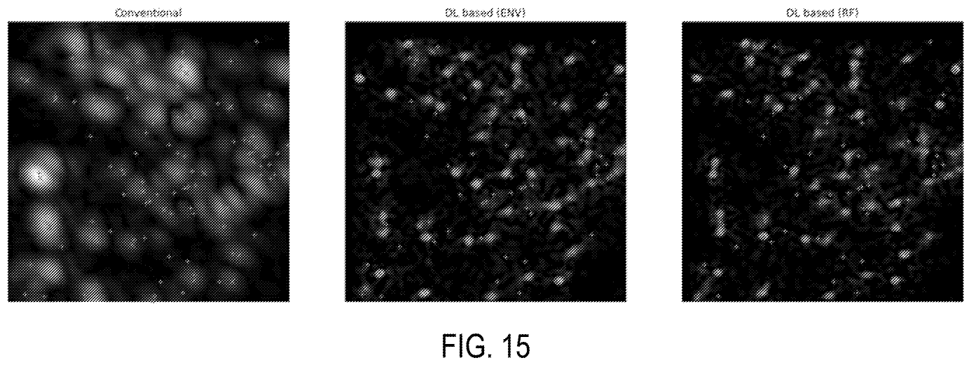

FIG. 15 shows examples of localization results using Field-II simulation (intensity envelope and RF) trained network: The conventional method uses normalized-cross-correlation to find regions that match the estimated PSF of the imaging system and uses the center of mass of each region as the bubble location. The two deep learning assisted methods use Field-II simulated envelope and RF data to train neural networks that deblurs input ultrasound data to smaller, sharper bubbles, and find the bubble locations using regional maxima. The red markers (x) in the figures indicate predicted bubble locations using each method while the green markers (+) are the true locations.

Localization scores are defined using the distance of a predicted bubble location to the nearest true location. A localization is considered correct if the distance is within a tenth of the wavelength. A prediction is considered a false localization if it is more than half a wavelength apart from the nearest ground truth location. A bubble is considered to be completely missed if it is more than half a wavelength apart from the nearest predicted location. Anything in between is accounted for in the localization error. The experiments were performed on a simulated validation set of 10,000 images, with 100 instances for each of the bubble concentrations from 1 to 100.

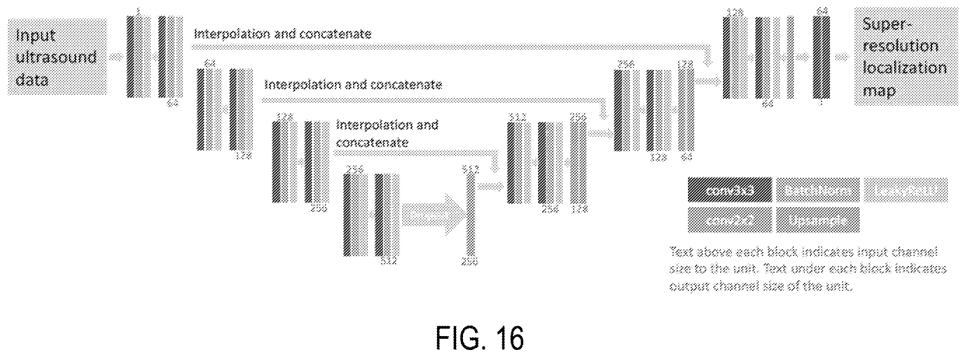

FIG. 16 demonstrates a specific, non-limiting example of a NN based on the U-Net architecture for generating super-resolution localization maps. The example NN is a convolutional neural network (“CNN”) architecture utilizing an encoder-decoder structure. The model contains a feature extraction path (the encoder) that maps the input ultrasound data onto a high-dimensional feature space, and a reconstruction path (the decoder) that extracts microbubble locations from the embedded feature map. Each feature extraction block contains two 3×3-kernel size convolution-batch normalization-activation units. Each reconstruction block starts with a convolution layer of kernel size 2×2 that halves the size of the feature space. The output of the corresponding feature extraction block can be upsampled to match the spatial dimension of the output of the 2×2 convolution layer before they were stacked along the channel axis. The stacked data then go through convolution-batch normalization-activation units and are upsampled by a factor of 2. Dropout was implemented in the bottleneck layer to prevent overfitting. The final output block contains two 2D convolution layers with 3×3 kernel size. A leaky ReLU can be used as the activation function.

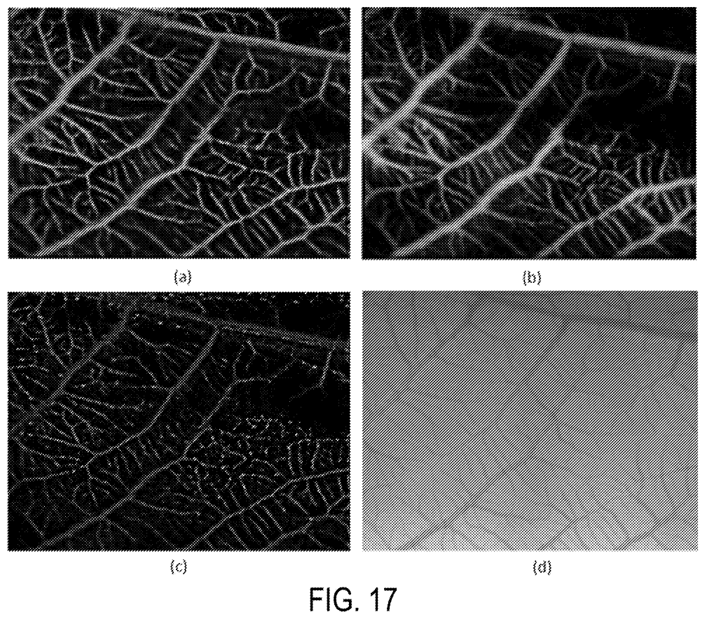

FIG. 17 shows super-resolved vessel maps of the CAM surface vessels obtained by accumulating locations of microbubbles in 1,600 frames (corresponding to 1.6 seconds) using envelope (“ENV”), RF, and the conventional methods. The region within this figure shows that deep learning-assisted methods detected smoother and more intact microvasculature of the CAM. In the illustrated example, deep learning-ENV was also capable of detecting vessels missing in the conventional method where bubble signals were weak. The microbubble localization steps presented in this figure represent a non-limiting example of process block 1112 feeding into process block 1118 to produce a SR vessel map.

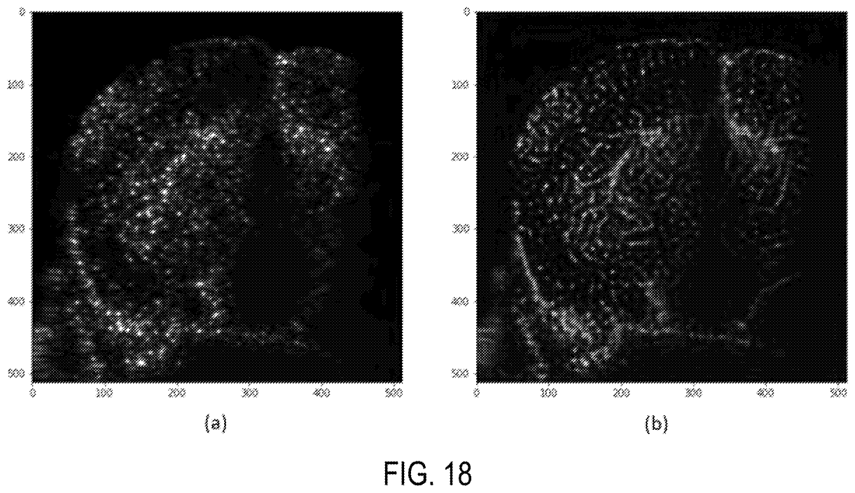

FIG. 18 shows an example of real-time display of blood flow in a mouse brain, corresponding to process block 1108. The figure on the left is the image of deep learning-processed, deblurred microbubbles overlaid on the original ultrasound image. The figure on the right is the display of super-resolution-like brain microvasculature obtained by accumulating 50 consecutive frames of deblurred microbubble images, as in process block 1110. The processing speed of forward propagation in the NN was measured at approximately 150 nanoseconds per pixel, which is substantially faster than conventional ULM processing requirements.

Training a NN to achieve robust SMV generally requires temporal information in the data class used for processing to adequately capture velocity and flow dynamics. All data acquisition procedures described above that provide temporal information can be used to train an SMV network. As another non-limiting example, either one of or a combination of in vivo data, ex vivo data, or vascular network generation/simulation can be combined with synthetic data generation to produce a physiologically relevant training dataset for NN training. The combination of experimental data and synthetic data is not exclusive to the SMV training process, as it can also apply to training of other forms of NN super-resolution ultrasound microvessel imaging and is included here for illustrative purposes. The applicable imaging modalities can include, but are not limited to, any of the example in vivo and ex vivo data acquisitions described above, and do not necessarily require concurrent ultrasound imaging or any combination of modalities.

As a non-limiting example of in vivo data, ex ovo chicken embryo chorioallantoic membranes, or any exposed shallow vascular bed, can undergo optical imaging as described in the above sections, to generate a large dataset of superficial vascular bed images. Other invasive optical imaging methods that insert optical sensors into the tissue can also be used to obtain vascular images of deep tissues. The vascular component can then be extracted using any combination of image processing steps, including, but not limited to manual segmentation, single or multistep thresholding, adaptive segmentation, and seeded region growing, to produce estimates of the blood vessel geometry. This experimentally determined blood vessel architecture can then be used as input into the synthetic data generation unit by simulating microvasculature through assignment of point scatterer locations within vessel lumen. Temporal data can be generated by displacing or otherwise moving these point scatterers via locally assigned flow speeds at each time instant.

Methods for determining local flow speed can include, but are not limited to, experimental determination, numerical simulation via blood flow models (non-limiting examples include Poiseuille flow, laminar flow, plug flow, capillary action, Bernoulli's principle, solutions to the Navier-Stokes equations, partially-developed flow, turbulent flow, diffusive and convective models with Newtonian and/or non-Newtonian fluids, linear flow, flow through arbitrary cross-sections, and minimization of wall-shear stress), or finite element modeling of particle flow through geometry. As an alternative approach, one can use data directly acquired from optical imaging for training purposes. For example, microbubbles can be fluorescently labeled and injected in vivo, followed by optical imaging of the vasculature that contains the microbubble signal. In this case the microbubble signal (with temporal flow information) can be directly used for training purposes.

As another non-limiting example, ex vivo tissues can be extracted from relevant experimental organs to serve as a geometrical model for synthetic flow simulations. This results in either a 2D or 3D vascular network geometry which can serve as the locations for point scatterer assignment in the synthetic data unit and can be used as input into the local flow estimation models discussed above to generate temporal flow information for network training. As another non-limiting example, blood vessel geometry can be simulated or modeled using any combination of fractal branching, 2D or 3D random walks, interactive geometry, artists depiction, fine element modeling/simulation, coupled blood flow oxygen transport models, wall-shear stress simulation, reduction of work simulations, diffusion of ligand models (e.g.: VEGF), or any computer-aided mathematical simulation of the biological processes and systems which generate and govern the structure and function of blood vessels and vascular networks. These simulated or otherwise generated vessel geometries and/or velocities can then serve as an input for generating synthetic data. Datasets that are generated using this protocol can produce a large amount of high quality spatial-temporal microbubble data that is particularly well suited to training a NN that does not require explicit microbubble localization to estimate blood flow velocity and vascular quantification, such as SMV.

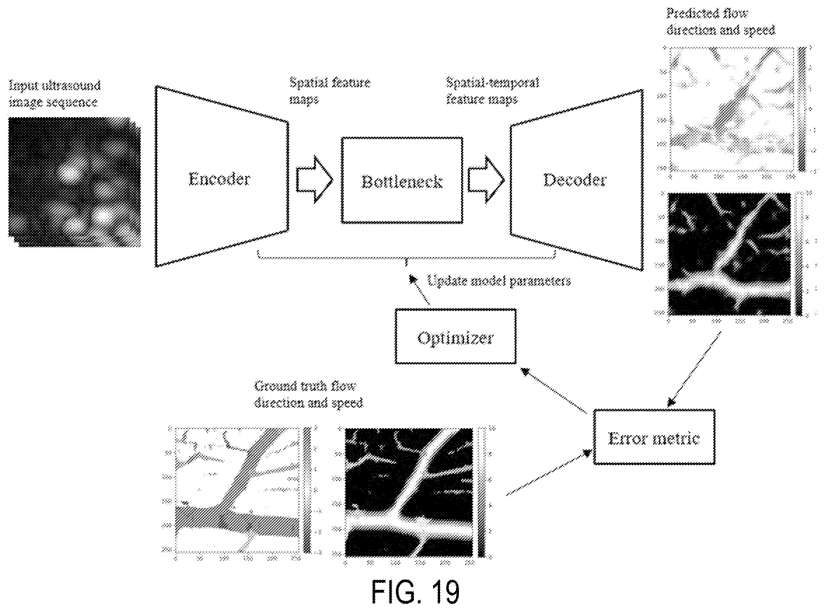

FIG. 19 shows a non-limiting example of the spatial-temporal DL training procedure for SMV using simulated MB flow data. The DL model includes an encoder block, which is a convolutional NN that extracts spatial information from each of the individual frames in the MB image sequence. The spatial feature maps for each frame are stacked together to feed into the bottleneck block, composed of long short-term memory (“LSTM”) units, that enables prediction based on both spatial and temporal information. The bottleneck block outputs spatial-temporal feature maps for each sequence of input data. The decoder block, which again is a convolutional NN, performs prediction based on the spatial-temporal feature maps. The final prediction output contains two channels: the predicted flow velocity magnitudes and the predicted flow velocity directions. The difference between the predicted output and the ground truth can be evaluated based on an error metric. Error metrics can be calculated with one or the following methods, or combinations thereof: L1 norm, L2 norm, structural similarity (“SSIM”), and multi-scale structural similarity (“MS-SSIM”). Further, the error metrics can be calculated in either the original data space (e.g., ultrasound images), or in a latent space (e.g., latent vectors extracted from an autoencoder), or the combination of both. The gradient of this difference can be calculated and used by an optimizer to update the model parameters.

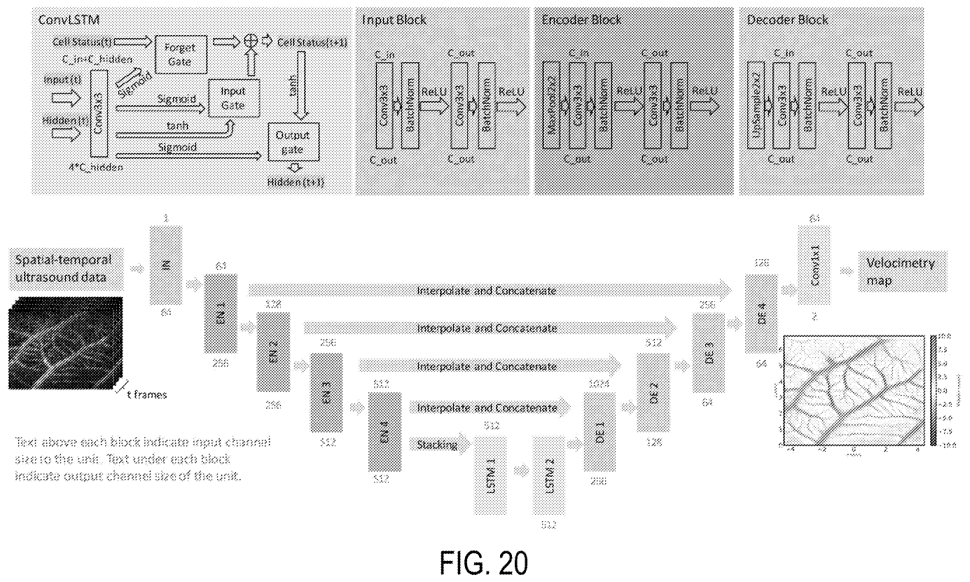

FIG. 20 demonstrates a specific, non-limiting example of a NN LSTM architecture, which satisfies the requirements for a spatial-temporal DL of 2D+temporal or 3D+temporal datasets for SMV. The input block contains two convolution layers with 3*3 kernel size, each followed by a batch normalization layer and a Rectified Linear Unit (ReLU) activation function. The encoder block contains a 2*2 max pooling layer and two 3*3 convolution layers, each followed by a batch normalization layer and ReLU activation function. Each encoder block reduces all spatial dimensions of its input by a factor of two and expands the feature dimension by a factor of two. The convolutional-LSTM block takes a new input and the previous hidden state output as its inputs, while maintaining an internal cell status variable. The inputs will first go through a 3*3 convolution layer. The convolution layer output will be split along the channel dimension into four parts, where three will go through a sigmoid activation function and operate as the forget gate, the input gate, and the output gate. A hyperbolic tangent (tanh) function will be applied to the remaining part. The forget gate determines which part of the previous cell status will be discarded. The input gate determines whether parts of the new input contribute to updating the cell status. A tanh activation function will be applied to the new cell status. Finally, the output gate determines how the cell status propagates to the output. The convolutional LSTM block will reduce the input sequence along the temporal dimension. The decoder block is constructed similarly as the encoder block, with the max pooling layer replaced by spatial upsampling layer. Each decoder block will expand the spatial dimension of its input by 2 and reduce the channel dimension by 2. The final output block is a 3*3 convolution layer with output channel size of 2, representing super-resolved maps of velocity and direction of flow.

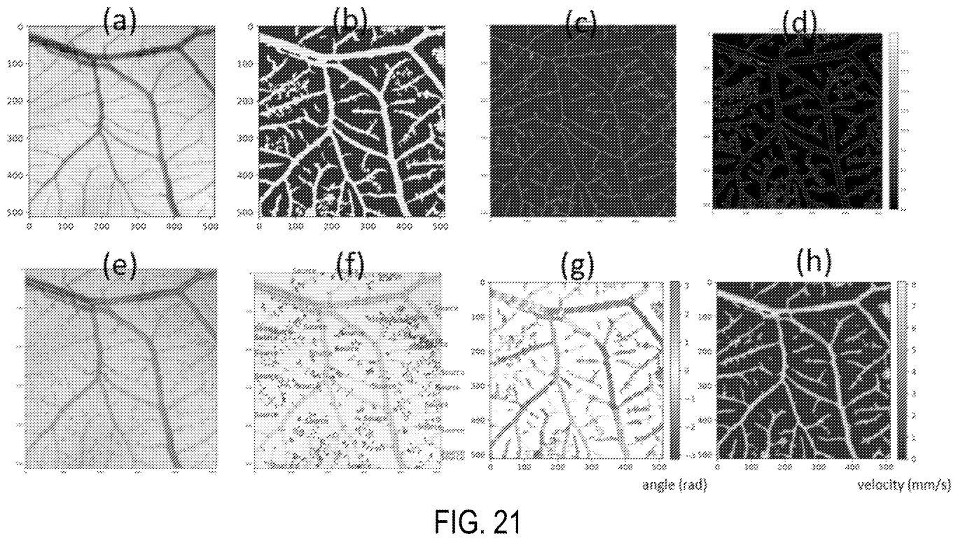

FIG. 21 provides a non-limiting example procedure of using in vivo optical images of CAM surface vessels (a) and numerical simulation data to generate a model for the blood flow of the vascular system. Adaptive thresholding was performed on the optical image to provide a binary segmentation (b) of the vessel structure. The binary segmentation map was processed using medial-axis skeletonization method, which results in a skeleton image (c), as well as a distance map (d) that can be used to obtain the radius of each vessel segment. The skeleton was then converted to a graph (e) of edges that represent vessel segments connected by nodes. The direction of blood flow (f) in each vessel segments can be inferred from the connectivity information and the vessel radius on the segments. Flow velocity on each vessel segment was assigned based on flow velocity of a vessel with the same size measured by conventional ULM. Such models can be used to generate 2-D spatial, 1-D temporal MB data as training input. 3-D spatial, 1-D temporal is also possible using this experimental setup but are not demonstrated here. Initially, MBs will be randomly distributed within the vessels based on the estimated volume of each vessel segment. For each time step, the positions of the MBs will be updated based on the flow velocity assigned to the vessel segment it is currently in, as well as the distance between the MB and the medial axis of the segment (i.e., center of the vessel). MBs that reach a junction point of multiple vessel segments will branch into one of the neighboring vessel segments. MBs reaching the end of a vessel segment with no neighboring vessels will be removed. The spatial coordinates of the MBs will be recorded for each update and used by an ultrasound simulation program to simulate consecutive frames of ultrasound MB motion data. The flow direction and the flow speed will be mapped back to the binary segmentation map to provide ground truth for DL training ((g) and (h)).

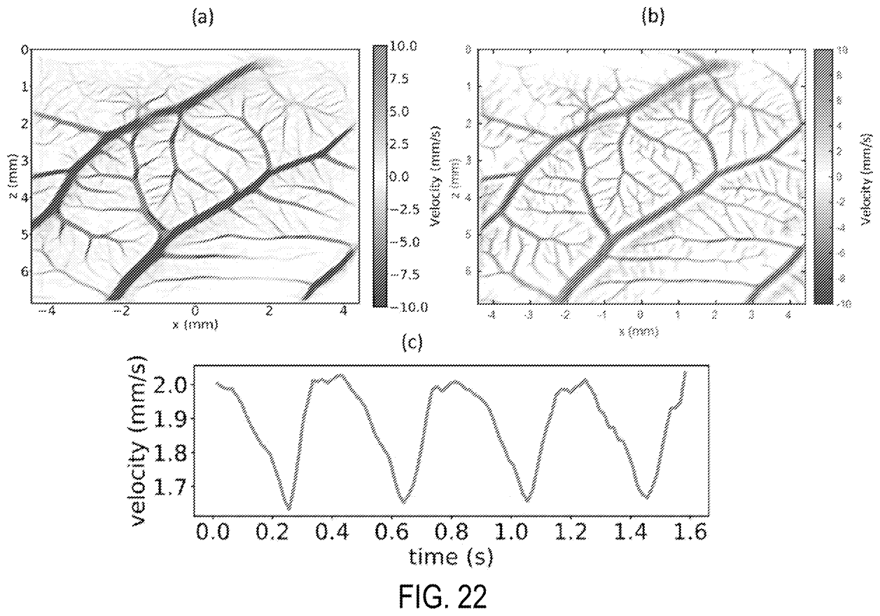

FIG. 22 (a) shows the blood flow velocity map of a CAM surface vessel obtained using DL-based ultrasound microbubble velocimetry, SMV, corresponding to block 316. The result was obtained from 1,600 consecutive frames (1.6 s) of ultrasound MB data. (b) shows the blood flow velocity map of the same CAM surface vessel obtained by conventional ULM, with 32,000 frames (32 s) of ultrasound MB data. The DL-based method was able to obtain results of comparable quality with the conventional method with 20 times less data. Moreover, the total processing time of the DL based method was around 7 minutes, while the conventional ULM processing takes a few hours. (c) shows a curve of average velocity of the vessels measured by DL-based velocimetry over time. Fluctuation of the blood flow speed can be observed from the curve with a behavior that matches the typical cardiac cycle of a chicken embryo at this stage of development.

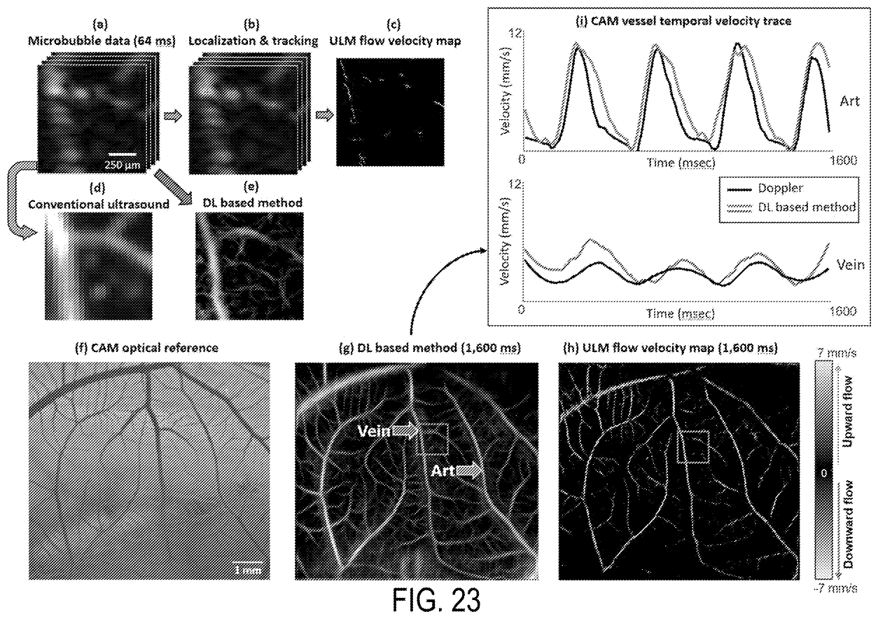

FIGS. 23A-23I illustrate an example of processing microbubble signal data using the systems and methods described in the present disclosure, as compared to processing those same signals with conventional ULM techniques. FIG. 23A shows a short data segment (64 ms) of unprocessed microbubble data. This data was processed with conventional ULM localization and tracking, as shown in FIG. 23B, resulting in a very sparse flow velocity map where it is difficult to visualize the microvessel anatomy, as seen in FIG. 23C. Direct accumulation of MB signal data, as illustrated in FIG. 23D, reveals diffraction-limited vessel structure. The microbubble signal data of FIG. 23A were also processed with the deep learning-based methods described in the present disclosure, which as illustrated in FIG. 23E allowed for super-resolution velocity estimation with more structural connectivity than conventional ULM. The orange color in FIGS. 23C and 23E indicates flow toward the transducer, and blue indicates flow away from the transducer. FIGS. 23F-23H demonstrate the deep learning-based method and ULM performance comparison on long data segments with a large field-of-view. FIG. 23I illustrates that the deep learning-based method shows distinct pulsatility features in local regions sampled from feeding (e.g., artery labeled “Art” in FIG. 23G) and draining (e.g., vein labeled “Vein” in FIG. 23G) vasculature, which matches conventional Doppler velocity measurements.

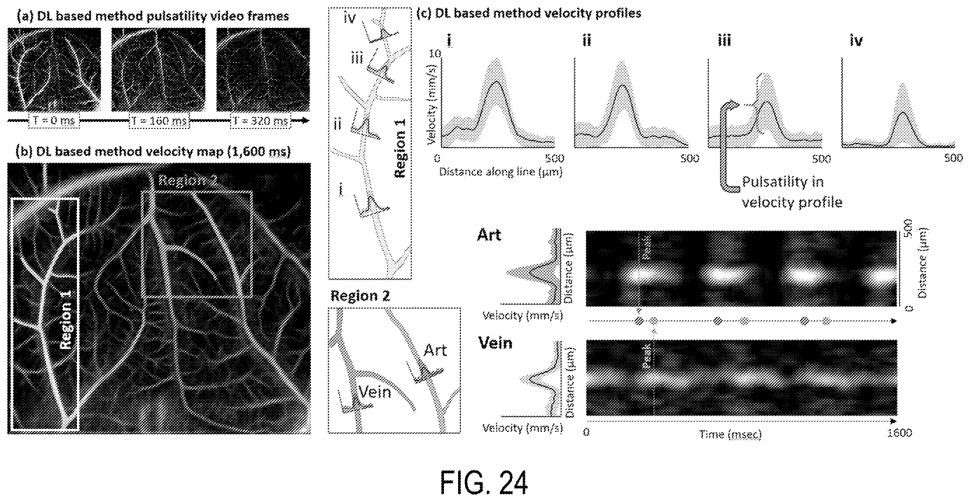

FIGS. 24A-24C demonstrate a detailed CAM flow dynamics analysis using the deep learning-based methods described in the present disclosure for estimating velocity. FIG. 24A shows video frames taken from a deep learning-based method pulsatility video, which demonstrates cycling pulsatility and different flow dynamics for feeding and draining vessels. FIG. 24B shows an accumulated deep learning-based velocity map, which was generated using 1,600 consecutive frames (1.6 s) of ultrasound microbubble signal data, with two selected regions. As shown in FIG. 24C, Region 1 demonstrates a gradual decrease in peak velocity and pulsatility in velocity profiles along the vessel length (i to iv), while Region 2 demonstrates a phase delay in the peak velocity estimate for vessels labeled Art and Vein.

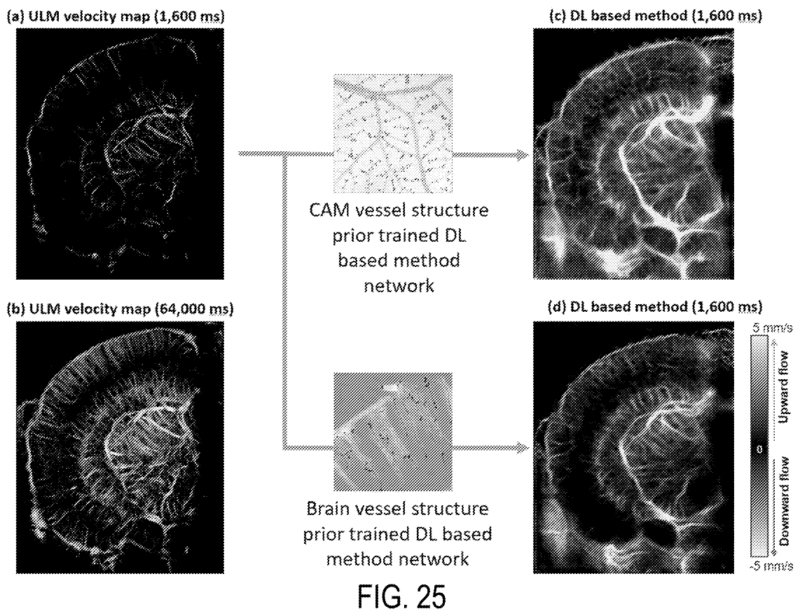

FIGS. 25A-25D demonstrate the influence of different vessel structure priors on deep learning-based ultrasound microbubble velocimetry. FIG. 25A illustrates conventional ULM localization and tracking for a 1600 ms mouse brain dataset, which was only able to sparsely populate the image with velocity estimates. FIG. 25B shows that, due to the inefficient use of microbubble signals, a much longer acquisition time (e.g., 64,000 ms) is required to reconstruct the majority of brain vasculature using ULM techniques. FIG. 25C shows that the deep learning-based methods described in the present disclosure (e.g., a deep learning model trained with CAM simulation data) had acceptable performance for larger subcortical vessels. FIG. 25D shows an example in which a mouse-brain prior trained deep learning-based method network had improved performance in cortical regions, while maintaining the ability to reconstruct subcortical features.

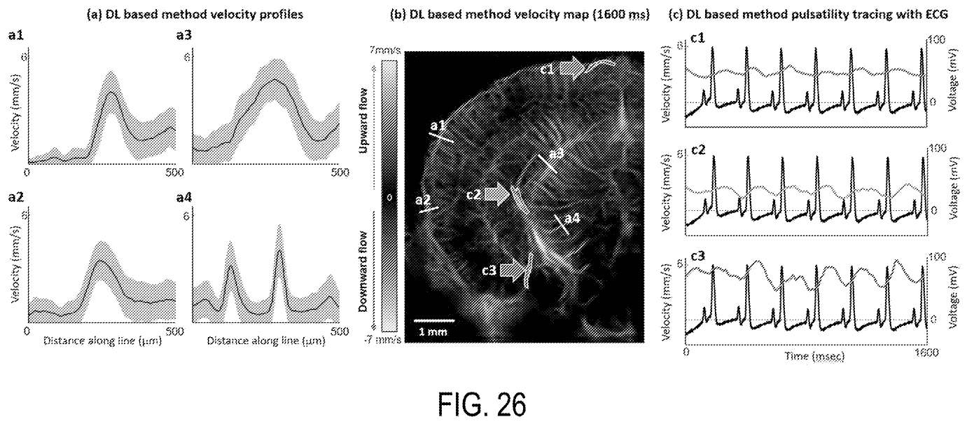

FIGS. 26A-26C demonstrates an example of applying the deep learning-based methods described in the present disclosure to mouse brain vessel velocimetry, validated with an electrocardiogram. FIG. 26A shows velocity profiles generated from cortical (a1 and a2) and subcortical (a3 and a4) vasculature, taken from the marked locations shown in FIG. 26B, which depicts the deep learning-based based velocimetry map of a mouse brain. FIG. 26C shows velocity traces of cortical vessel (c1) and subcortical vessel (c2 and c3) segmentations in comparison to electrocardiogram measurements. The velocity traces show a good correspondence to the external validation method.

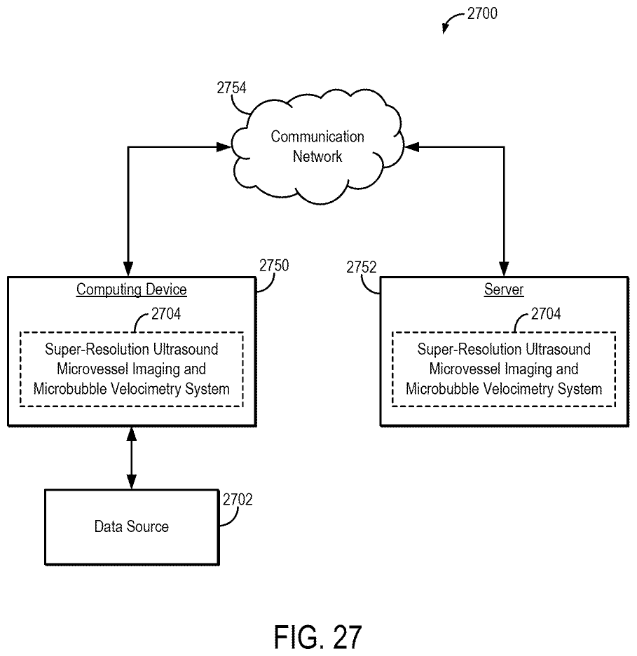

Referring now to FIG. 27, an example of a system 2700 for super-resolution ultrasound microvessel imaging and microbubble velocimetry in accordance with some embodiments of the systems and methods described in the present disclosure is shown. As shown in FIG. 27, a computing device 2750 can receive one or more types of data (e.g., ultrasound signal data, optical image data, training data, neural network parameter data) from data source 2702, which may be an ultrasound data source, an optical image data source, and the like. In some embodiments, computing device 2750 can execute at least a portion of a super-resolution ultrasound microvessel imaging and microbubble velocimetry system 2704 to generate super-resolution microvessel images and/or microvessel velocimetry maps (e.g., localization maps, SR vessel maps, SR flow speed maps, accumulated SR-like images) from data received from the data source 2702.

Additionally or alternatively, in some embodiments, the computing device 2750 can communicate information about data received from the data source 2702 to a server 2752 over a communication network 2754, which can execute at least a portion of the super-resolution ultrasound microvessel imaging and microbubble velocimetry system 2704. In such embodiments, the server 2752 can return information to the computing device 2750 (and/or any other suitable computing device) indicative of an output of the super-resolution ultrasound microvessel imaging and microbubble velocimetry system 2704.

In some embodiments, computing device 2750 and/or server 2752 can be any suitable computing device or combination of devices, such as a desktop computer, a laptop computer, a smartphone, a tablet computer, a wearable computer, a server computer, a virtual machine being executed by a physical computing device, and so on. The computing device 2750 and/or server 2752 can also reconstruct images from the data.

In some embodiments, data source 2702 can be any suitable source of image data (e.g., measurement data, images reconstructed from measurement data), such as an ultrasound imaging system, another computing device (e.g., a server storing image data), and so on. In some embodiments, data source 2702 can be local to computing device 2750. For example, data source 2702 can be incorporated with computing device 2750 (e.g., computing device 2750 can be configured as part of a device for capturing, scanning, and/or storing images). As another example, data source 2702 can be connected to computing device 2750 by a cable, a direct wireless link, and so on. Additionally or alternatively, in some embodiments, data source 2702 can be located locally and/or remotely from computing device 2750, and can communicate data to computing device 2750 (and/or server 2752) via a communication network (e.g., communication network 2754).

In some embodiments, communication network 2754 can be any suitable communication network or combination of communication networks. For example, communication network 2754 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, and so on. In some embodiments, communication network 2754 can be a local area network, a wide area network, a public network (e.g., the Internet), a private or semi-private network (e.g., a corporate or university intranet), any other suitable type of network, or any suitable combination of networks. Communications links shown in FIG. 27 can each be any suitable communications link or combination of communications links, such as wired links, fiber optic links, Wi-Fi links, Bluetooth links, cellular links, and so on.

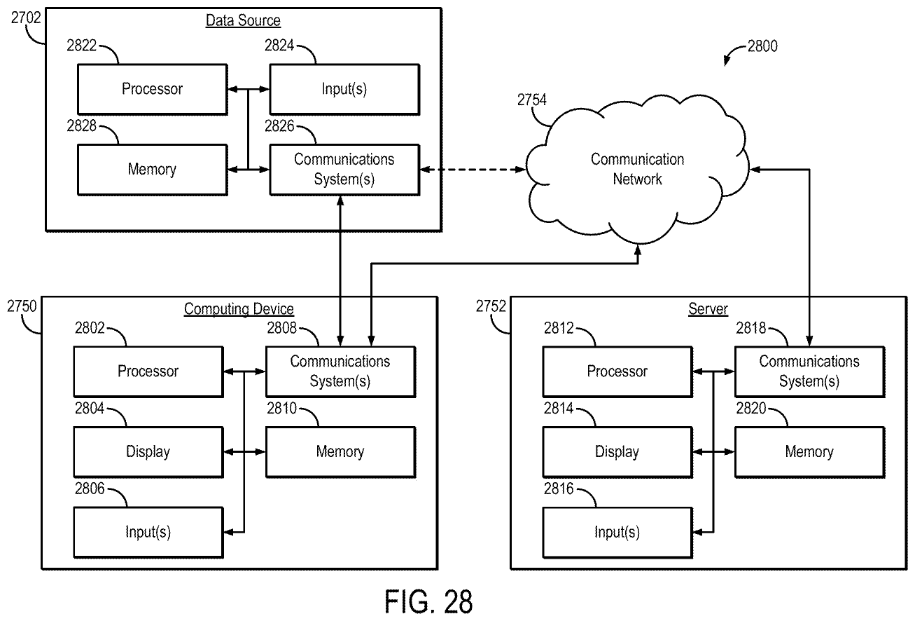

Referring now to FIG. 28, an example of hardware 2800 that can be used to implement data source 2702, computing device 2750, and server 2752 in accordance with some embodiments of the systems and methods described in the present disclosure is shown. As shown in FIG. 28, in some embodiments, computing device 2750 can include a processor 2802, a display 2804, one or more inputs 2806, one or more communication systems 2808, and/or memory 2810. In some embodiments, processor 2802 can be any suitable hardware processor or combination of processors, such as a central processing unit (“CPU”), a graphics processing unit (“GPU”), and so on. In some embodiments, display 2804 can include any suitable display devices, such as a computer monitor, a touchscreen, a television, and so on. In some embodiments, inputs 2806 can include any suitable input devices and/or sensors that can be used to receive user input, such as a keyboard, a mouse, a touchscreen, a microphone, and so on.

In some embodiments, communications systems 2808 can include any suitable hardware, firmware, and/or software for communicating information over communication network 2754 and/or any other suitable communication networks. For example, communications systems 2808 can include one or more transceivers, one or more communication chips and/or chip sets, and so on. In a more particular example, communications systems 2808 can include hardware, firmware and/or software that can be used to establish a Wi-Fi connection, a Bluetooth connection, a cellular connection, an Ethernet connection, and so on.

In some embodiments, memory 2810 can include any suitable storage device or devices that can be used to store instructions, values, data, or the like, that can be used, for example, by processor 2802 to present content using display 2804, to communicate with server 2752 via communications system(s) 2808, and so on. Memory 2810 can include any suitable volatile memory, non-volatile memory, storage, or any suitable combination thereof. For example, memory 2810 can include RAM, ROM, EEPROM, one or more flash drives, one or more hard disks, one or more solid state drives, one or more optical drives, and so on. In some embodiments, memory 2810 can have encoded thereon, or otherwise stored therein, a computer program for controlling operation of computing device 2750. In such embodiments, processor 2802 can execute at least a portion of the computer program to present content (e.g., images, user interfaces, graphics, tables), receive content from server 2752, transmit information to server 2752, and so on.

In some embodiments, server 2752 can include a processor 2812, a display 2814, one or more inputs 2816, one or more communications systems 2818, and/or memory 2820. In some embodiments, processor 2812 can be any suitable hardware processor or combination of processors, such as a CPU, a GPU, and so on. In some embodiments, display 2814 can include any suitable display devices, such as a computer monitor, a touchscreen, a television, and so on. In some embodiments, inputs 2816 can include any suitable input devices and/or sensors that can be used to receive user input, such as a keyboard, a mouse, a touchscreen, a microphone, and so on.

In some embodiments, communications systems 2818 can include any suitable hardware, firmware, and/or software for communicating information over communication network 2754 and/or any other suitable communication networks. For example, communications systems 2818 can include one or more transceivers, one or more communication chips and/or chip sets, and so on. In a more particular example, communications systems 2818 can include hardware, firmware and/or software that can be used to establish a Wi-Fi connection, a Bluetooth connection, a cellular connection, an Ethernet connection, and so on.

In some embodiments, memory 2820 can include any suitable storage device or devices that can be used to store instructions, values, data, or the like, that can be used, for example, by processor 2812 to present content using display 2814, to communicate with one or more computing devices 2750, and so on. Memory 2820 can include any suitable volatile memory, non-volatile memory, storage, or any suitable combination thereof. For example, memory 2820 can include RAM, ROM, EEPROM, one or more flash drives, one or more hard disks, one or more solid state drives, one or more optical drives, and so on. In some embodiments, memory 2820 can have encoded thereon a server program for controlling operation of server 2752. In such embodiments, processor 2812 can execute at least a portion of the server program to transmit information and/or content (e.g., data, images, a user interface) to one or more computing devices 2750, receive information and/or content from one or more computing devices 2750, receive instructions from one or more devices (e.g., a personal computer, a laptop computer, a tablet computer, a smartphone), and so on.

In some embodiments, data source 2702 can include a processor 2822, one or more inputs 2824, one or more communications systems 2826, and/or memory 2828. In some embodiments, processor 2822 can be any suitable hardware processor or combination of processors, such as a CPU, a GPU, and so on. In some embodiments, the one or more inputs 2824 are generally configured to acquire data, images, or both, and can include an ultrasound imaging system. Additionally or alternatively, in some embodiments, one or more inputs 2824 can include any suitable hardware, firmware, and/or software for coupling to and/or controlling operations of an ultrasound imaging system. In some embodiments, one or more portions of the one or more inputs 2824 can be removable and/or replaceable.

Note that, although not shown, data source 2702 can include any suitable inputs and/or outputs. For example, data source 2702 can include input devices and/or sensors that can be used to receive user input, such as a keyboard, a mouse, a touchscreen, a microphone, a trackpad, a trackball, and so on. As another example, data source 2702 can include any suitable display devices, such as a computer monitor, a touchscreen, a television, etc., one or more speakers, and so on.

In some embodiments, communications systems 2826 can include any suitable hardware, firmware, and/or software for communicating information to computing device 2750 (and, in some embodiments, over communication network 2754 and/or any other suitable communication networks). For example, communications systems 2826 can include one or more transceivers, one or more communication chips and/or chip sets, and so on. In a more particular example, communications systems 2826 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 2828 can include any suitable storage device or devices that can be used to store instructions, values, data, or the like, that can be used, for example, by processor 2822 to control the one or more inputs 2824, and/or receive data from the one or more inputs 2824; to images from data; present content (e.g., images, a user interface) using a display; communicate with one or more computing devices 2750; and so on. Memory 2828 can include any suitable volatile memory, non-volatile memory, storage, or any suitable combination thereof. For example, memory 2828 can include RAM, ROM, EEPROM, one or more flash drives, one or more hard disks, one or more solid state drives, one or more optical drives, and so on. In some embodiments, memory 2828 can have encoded thereon, or otherwise stored therein, a program for controlling operation of data source 2702. In such embodiments, processor 2822 can execute at least a portion of the program to generate images, transmit information and/or content (e.g., data, images) to one or more computing devices 2750, receive information and/or content from one or more computing devices 2750, receive instructions from one or more devices (e.g., a personal computer, a laptop computer, a tablet computer, a smartphone, etc.), and so on.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “unit,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

The present disclosure has described one or more preferred embodiments, and it should be appreciated that many equivalents, alternatives, variations, and modifications, aside from those expressly stated, are possible and within the scope of the invention.

1. A method for super-resolution microvessel imaging using an ultrasound system, the method comprising:
acquiring ultrasound signal data from a subject using the ultrasound system;
accessing, with a computer system, a neural network that has been trained on training data to estimate at least one of super-resolution microvessel image data or super-resolution ultrasound velocimetry data from ultrasound signals;
inputting the ultrasound signal data to the neural network, via the computer system, generating output data as at least one of super-resolution microvessel image data or super-resolution ultrasound velocimetry data; and
providing the at least one of the super-resolution microvessel image data or the super-resolution ultrasound velocimetry data to a user via the computer system.

2. The method of claim 1, wherein the training data and the input data comprise contrast-enhanced spatiotemporal ultrasound signal data.

3. The method of claim 1, wherein the training data and the input data comprise non-contrast-enhanced spatiotemporal ultrasound signal data.

4. The method of claim 1, wherein the neural network comprises at least one of a pre-trained neural network and an online trained neural network.

5. The method of claim 4, wherein the neural network is a pre-trained neural network and the training data comprise at least one of synthetic training data, in vitro training data, ex vivo training data, or in vivo training data.

6. The method of claim 5, wherein the training data comprise synthetic training data generated by computer simulation of spatiotemporal microbubble signals observed using ultrasound.

7. The method of claim 6, wherein the computer simulation comprises at least one of a direct convolution between a microbubble location and one of point-spread-function (PSF) or impulse response (IR) of the ultrasound system.

8. The method of claim 7, wherein the computer simulation is implemented with the computer system using a Field II simulation, a K-wave simulation, or a generative neural network.

9. The method of claim 5, wherein the training data comprise in vitro training spatiotemporal data acquired from at least one of a tissue-mimicking phantom and a point target.

10. The method of claim 5, wherein the training data comprise ex vivo spatiotemporal training data acquired using ultrasound imaging of biological tissue with contrast microbubbles.

11. The method of claim 1, wherein training data comprise in vivo spatiotemporal training data acquired by at least one of ultrasound imaging of in vivo tissue or simultaneous optical and ultrasound imaging of in vivo tissue.

12. The method of claim 1, wherein accessing the neural network with the computer system comprises training the neural network with the computer system by:
accessing training data with the computer system;
training the neural network on the training data; and
storing the trained neural network.

13. The method of claim 12, wherein accessing the training data comprise acquiring spatiotemporal imaging data from an in vivo vascular bed.

14. The method of claim 13, wherein the spatiotemporal imaging data comprise spatiotemporal acoustic imaging data.

15. The method of claim 14, wherein the spatiotemporal acoustic imaging data are indicative of at least one of anatomical information, dynamic information, or contrast-enhanced information.

16. The method of claim 14, wherein the spatiotemporal imaging data further include spatiotemporal optical imaging data.

17. The method of claim 16, wherein the spatiotemporal optical imaging data are indicative of at least one of anatomical information, dynamic information, or contrast-enhanced information.

18. The method of claim 16, wherein the spatiotemporal acoustic imaging data and the spatiotemporal optical imaging data are spatially coregistered.

19. The method of claim 16, wherein the spatiotemporal acoustic imaging data and the spatiotemporal optical imaging data are synchronously acquired from the in vivo vascular bed.

20. The method of claim 16, further comprising synchronizing the spatiotemporal acoustic imaging data acquisition and the spatiotemporal optical imaging data acquisition in space and time to capture matched data.

21. The method of claim 20, wherein the spatiotemporal training data are based on the matched data.

22. The method of claim 12, wherein training the neural network comprises:
administering scout microbubbles into a subject to collect microbubble signal data;
identifying a scouting microbubble signal in the microbubble signal data; and
using the scouting microbubble signal to tune the trained neural network.

23. The method of claim 1, wherein the super-resolution ultrasound microvessel image data comprise a sharpened microbubble image, and providing the super-resolution microvessel image data via the computer system includes accumulating sharpened microbubble signals to achieve real-time display of tissue microvasculature.

24. The method of claim 1, wherein the super-resolution ultrasound microvessel image data comprises locations of microbubbles, and providing the super-resolution microvessel image data via the computer system includes pairing, tracking and accumulating a microbubble signal to generate super-resolution vessel maps and super-resolution flow maps.

25. The method of claim 1, wherein providing the super-resolution microvessel image data via the computer system includes comprises performing fast localization and tracking to construct a super-resolution image.

26. The method of claim 1, wherein the super-resolution ultrasound velocimetry data comprises at least one flow velocity map indicating flow velocity of microbubbles.

27. The method of claim 26, wherein the at least one flow velocity map is generated without localization of the microbubbles.

28. A method for training a neural network for super-resolution ultrasound microvessel imaging, the method comprising:
providing a chorioallantoic membrane (CAM) assay;
performing simultaneous imaging of the CAM assay with an optical imaging system and an acoustic imaging system, generating optical CAM assay image data and acoustic CAM assay image data; and
assembling, with a computer system, a training data set based on the optical CAM assay image data and the acoustic CAM assay image data; and
training a neural network using the training data set.

29. The method of claim 28, further comprising administering a microbubble contrast agent to the CAM assay before performing the simultaneous imaging of the CAM assay.

30. The method of claim 29, wherein the microbubble contrast agent is an optically labeled microbubble contrast agent.

31. The method of claim 28, wherein performing the simultaneous imaging of the CAM assay comprises:
spatially registering an optical imaging system and an acoustic system; and
temporally synchronizing the optical imaging system and the acoustic imaging system.
